(19) United States
(12) Patent Application Publication
(10) Pub. No.: US 2021/0069377 A1
(43) Pub. Date: Mar. 11, 2021

(54) TISSUE SCAFFOLD

(71) Applicant: Matoke Holdings Limited, Southmoor, Abingdon Oxfordshire (GB)

(72) Inventors: David Kershaw, Southmoor, Abingdon Oxfordshire (GB); Paulo Bartolo, Southmoor, Abingdon Oxfordshire (GB); Carl Diver, Southmoor, Abingdon Oxfordshire (GB); Enes Aslan, Southmoor, Abingdon Oxfordshire (GB); Cian Vyas, Southmoor, Abingdon Oxfordshire (GB); Iain Elder, Southmoor, Abingdon Oxfordshire (GB)

(21) Appl. No.: 17/046,187

(22) PCT Filed: Apr. 11, 2019
(86) PCT No.: PCT/GB2019/051055

§ 371 Date: Oct. 8, 2020

(30) Foreign Application Priority Data
Apr. 11, 2018 (GB) 1805996.4

(51) Int. Cl. A61L 27/22; A61L 27/36; A61L 27/58; A61L 27/60; A61L 27/56; C12N 5/077

(52) U.S. Cl. CPC A61L 27/227; A61L 27/3637; A61L 27/58; A61L 27/36; C12N 2533/30; A61L 27/56; C12N 5/0653; A61L 2300/254; A61L 2400/12; A61L 27/60

(57) ABSTRACT

Tissue scaffold matrices, and methods of their use, are described. The matrices comprise an enzyme that is able to convert a substrate to release hydrogen peroxide and a substrate for the enzyme. The matrices may be impregnated with cells, such as stem cells. Also described are cell cultures, and methods for proliferating and/or differentiating cells.

This invention relates to matrices for use as tissue scaffolds, which promote cell attachment and proliferation and which have antimicrobial properties.

Hydrogen peroxide-generating compositions (e.g. SurgihoneyRO™) and wound dressings based on such compositions have been shown to provide remarkable antimicrobial properties, including against the growth and seeding of biofilms. Examples of such compositions and wound dressings are described in WO 2015/166197, WO 2016/083798 and WO 2016/124926. It has been appreciated that hydrogen peroxide can assist in the healing of damaged tissue by killing microbes that are preventing healing, and that hydrogen peroxide itself may assist in the body's natural tissue regeneration mechanisms. For example, reactive oxygen species have been demonstrated to promote wound healing by encouraging cellular repair processes and are involved in tail regeneration in tadpoles (N. R. Love et al. (2013)). However, there still remain challenges, particularly if the body's ability to heal is compromised.

Skin, for example, is the largest organ in the body protecting the internal tissues and organs from the external environment (JR. Diaset al, (2016)). However, skin is vulnerable to a wide range of injuries induced by acute trauma, burns, chronic wounds, cancer, and other dermatological diseases. Skin has a natural healing capacity, however, this can become compromised in chronic wound environments or if the affected area is too large such as in burns (R. F. Pereira, et al. (2016)). These non-healing wounds are painful, highly debilitating, and costly (F. Gottrup et al. (2010); K. Alexiadou et al. (2012)). Furthermore, such wounds are prone to microbial infection. Treatments are limited in these situations. The clinical golden standard relies on the use of autografts or allografts, but both present significant limitations. Autografts induce scarring at the donor site and lengthy hospital stays, while allografts present ethical and safety problems related to disease transmission and immune rejection.

To address such problems, the inventors have developed antimicrobial matrices that are able to support attachment, proliferation and differentiation of cells, and may thus assist in tissue engineering.

In a broad sense, the invention concerns a hydrogen peroxide-generating matrix, in particular, a matrix comprising an enzyme that is able to convert a substrate to release hydrogen peroxide and a substance that includes a substrate for the enzyme (and/or a purified precursor-substrate that can be converted to a substrate for the enzyme).

In some embodiments, the matrix may be a wound dressing or for use as a wound dressing. The invention may thus provide a wound dressing comprising the matrix. In some embodiments, the dressing may comprise a substrate on to which the matrix is applied.

Suitable substrates may include gauzes, bandages, tissues, films, gels, foams, hydrocolloids, alginates (such as AMS alginate foam or spun-bond alginate dressing), hydrogels, or polysaccharide pastes, granules, beads. The substrate may be foil, polypropylene or Cyrex®. The wound dressing may comprise a collagen or collagen-glycosaminoglycan matrix.

However, in preferred embodiments, the matrix is a tissue scaffold, is for use as a tissue scaffold or is for use in tissue engineering. A tissue scaffold is a material that permits desirable cellular interactions and contributes to the formation of new functional tissues. Cells may be impregnated or seeded onto or into the scaffold so that the scaffold supports tissue formation. A tissue scaffold may thus mimic the extracellular matrix of native tissue to allow cell attachment, proliferation and/or differentiation. It may provide a structure to which cells can adhere and multiply without causing toxicity or inhibition of cell replication. Tissue scaffolds may advantageously provide adequate porosity and pore size to facilitate cell seeding, diffusion of gases and nutrients, and vascularisation. Tissue scaffolds may also permit diffusion of cells or cell ingress. Biodegradability or bioabsorbability is a particularly desirable property since tissue scaffolds should preferably be absorbed by the surrounding tissues without the necessity of a surgical removal. Although biodegradability or bioabsorbability are desirable properties, tissue scaffolds should be resistant to rapid degradation to permit adequate time for promotion of cell attachment and proliferation.

The matrix of the invention may thus be an implant or suitable for implantation into a subject's tissue.

Tissue scaffolds are distinguished from wound dressings. Wound dressings are typically applied over a wound and may function to promote a moist environment, protect the wound against mechanical injury and microbial contamination. Some wound dressing may provide a more interactive role, for example, by modifying the wound chemical environment to assist in the healing process. However, wound dressings are typically replaced or removed during progression of the healing process, or after the tissue has healed.

Surprisingly, the inventors have discovered that matrices of the invention can support cell attachment proliferation and differentiation, whilst also being able to provide antimicrobial properties through hydrogen peroxide generation.

Surprisingly, the inventors have also found that matrices of the invention comprising hydrogen peroxide-generating compositions, such as SurgihoneyRO™, can increase cell proliferation compared to equivalent matrices not containing honey. Although not wishing to be bound by theory, it is believed that this may be a result of a more hydrophilic surface which may improve cell spreading and serum proteins from the media attaching in the correct configuration. The hydrogen peroxide may also contribute to promotion of cell growth.

According to the invention, there is provided³ matrix for use in tissue engineering or as a tissue scaffold, comprising an enzyme that is able to convert a substrate to release hydrogen peroxide and a substance that includes a substrate for the enzyme (and/or a purified precursor-substrate that can be converted to a substrate for the enzyme).

Preferably, the matrix does not comprise sufficient free water to allow the enzyme to convert the substrate. Hydrogen peroxide is generally unstable at ambient temperature. The lack of sufficient free water in some matrices of the invention may thus prevent the enzyme converting the substrate to release hydrogen peroxide, and may thus help to maintain the stability of the matrix for extended periods at ambient temperature. A matrix of the invention may include some water provided that there is not sufficient free water to allow the enzyme to convert the substrate.

The skilled person would understand that a matrix that does not comprise sufficient free water to allow the enzyme to convert the substrate, encompasses a composition that contains a trace amount (or low levels) of free water that may allow a trace amount (or low levels) of hydrogen peroxide to be produced.

Suitable amounts of water may vary depending on the precise components of the matrix. However, typically, a matrix of the invention will comprise less than 25% (by weight), preferably less than 20% (by weight) total water content, for example, 10%-19%, water. Matrices of the invention may comprise 19% or less (by weight) of water. Matrices of the invention may comprise 18% or less (by weight) of water.

Matrices of the invention may comprise substantially no hydrogen peroxide, or no detectable hydrogen peroxide. For example, hydrogen peroxide is preferably not detectable using a hydrogen peroxide test strip, such as a Quantofix® peroxide test stick (Sigma Aldrich, UK). For example, hydrogen peroxide may be present at a level less than 1 ppm or at a level less than 0.5 ppm. Hydrogen peroxide may be at a level less than 0.1 ppm. Hydrogen peroxide generation may only begin once there is sufficient free water present. For example, once the matrix has been implanted into a wound, the wound exudate may provide sufficient free water to begin hydrogen peroxide production.

Before dilution, hydrogen peroxide may be present at a concentration of 6 ppm or less, 5 ppm or less, 3 ppm or less, or 2 ppm or less. Hydrogen peroxide may be present in the matrix at a concentration of 120 μM or less, preferably 100 μM or less, more preferably 80 μM or less. Low or trace levels of hydrogen peroxide before dilution may advantageously improve the shelf life of compositions of the invention. Higher levels of hydrogen peroxide may result in loss of enzyme activity over time. This may be caused by oxidative damage to the enzyme by the hydrogen peroxide being produced.

Once the matrix is diluted, or contacted with ⁴ ater, hydrogen peroxide may be generated at substantial concentrations. At 1 hour, following a 1:1 dilution (by weight) with water, the level of hydrogen peroxide production may increase by a factor of at least 5, at least 10, at least 20, at least 50, at least 100, or at least 200. At 24 hours, following a 1:1 dilution (by weight) with water, the level of hydrogen peroxide production may increase by a factor of at least 5, at least 10, at least 20, at least 50, at least 100, or at least 200.

In matrices of the invention, the water activity (a_{w}) may be 0.8 or less, 0.7 or less, or 0.6 or less. For example, the water activity may be 0.2 to 0.8, for example 0.3 to 0.7 or 0.4 to 0.6. A low water activity may be advantageous in preventing microbial proliferation, and it may be advantageous in minimising hydrogen peroxide production prior to activation by dilution.

According to the invention, there is provided a matrix for use as a tissue scaffold, comprising an enzyme that is able to convert a substrate to release hydrogen peroxide and a substance that includes a substrate for the enzyme (and/or a purified precursor-substrate that can be converted to a substrate for the enzyme), wherein the matrix does not comprise sufficient free water to allow the enzyme to convert the substrate.

According to the invention, there is provided a matrix for use as a tissue scaffold, comprising an enzyme that is able to convert a substrate to release hydrogen peroxide and a substance that includes a substrate for the enzyme (and/or a purified precursor-substrate that can be converted to a substrate for the enzyme), wherein the matrix has a water activity of 0.8 or less.

Preferably, matrices of the invention comprise one or more fibers, such as one or more nanofibers.

Use of the term “nanofiber” herein refers to a fiber with a diameter less than or equal to 1000 nanometres (nm). However, matrices of the invention may comprise one or more microfibers in addition to, or as an alternative to, the one or more nanofibers. The one or more fibers may have a diameter less than or equal to 1 millimetre, less than or equal to 500 micrometres (μm), less than or equal to 50 μm or less than or equal to 10 μm. In some embodiments, the one or more fibers may have a diameter less than or equal to 100 nm. In some embodiments, the one or more fibers may have a maximum diameter less than or equal to 20 μm. The one or more fibers may have a diameter greater than or equal to 0.2 μm. The one or more fibers may have an average (mean) diameter of 1 to 10 μm. The one or more fibers may have an average (mean) diameter of 1 to 5 μm.

In some embodiments, the matrix may have a fiber diameter range within 5 to 500 nm, 10 to 500 nm, 50 to 300 nm or 100 to 250 nm,

In some embodiments, the matrix may have ⁵ mean fiber diameter of 5 to 500 nm, 10 to 500 nm, 50 to 300 nm, 100 to 250 nm or 100 to 200 nm.

The one or more fibers may form a mesh. The one or more fibers may be aligned or randomly oriented. Preferably, the fibers are randomly oriented.

Fibers, such as nanofibers, may be particularly suitable for use in a tissue scaffold. This is because they may provide a high surface to volume ratio, enabling better attachment of cells and enhanced vascularisation. They may also provide high porosity to ensure transmission of gases and nutrients through the matrix.

The inventors have established how characteristics of the matrix, such as the fiber diameter and pore size, can be manipulated. This is described in more detail below. The matrix may thus be adapted to be optimal for particular cell-types or particular tissues. References herein to pore size may refer to pore diameter.

Characteristics of scaffolds such as pore size, pore area and fiber diameter, may be calculated using appropriate readily-available software. For example, ND (“Nearest Distance”) is an lmageJ plugin that was developed to calculate the average size and distance between pores and their nearest neighbours in porous scaffolds (see Haeri et al. (2015)). DiameterJ is another example of an lmageJ plugin that can be used to measure pore parameters. Microscopic images of the scaffold (e.g. SEM images) may be used as input. Different geometric descriptors can be used to analyse pore size, such as pore equivalent diameter, pore maximum opening, inscribed circle diameter and pore equivalent area. Other methods known to the skilled person for measuring pore parameters include uCT, gravimetric method and porosimetry.

In some embodiments, the mean pore size is at least 0.1 μm, at least 0.25 μm, at least 0.5 μm, at least 1 μm, at least 2 μm, at least 5 μm, at least 10 μm, at least 20 μm, at least 50 μm, at least 100 μm or at least 200 μm.

In some embodiments, the mean pore size is 1000 μm or less, 750 μm or less, 500 μm or less, 250 μm or less, 100 μm or less, 50 μm or less, 25 μm or less, 10 μm or less, 5 μm or less, 2 μm or less, or 1 μm or less.

In some embodiments, the mean pore size is 0.1 μm to 1000 μm, 0.1 μm to 500 μm, 0.1 μm to 250 μm, 0.1 μm to 100 μm, 0.1 μm to 50 μm, 0.1 μm to 25 μm, 0.1 μm to 10 μm, 0.1 μm to 5 μm, 0.1 μm to 2 μm, or 0.1 μm to 1 μm.

In some embodiments, the mean pore size is 0.25 μm to 1000 μm, 0.25 μm to 500 μm, 0.25 μm to 250 μm, 0.25 μm to 100 μm, 0.25 μm to 50 μm, 0.25 μm to 25 μm. In some embodiments, the mean pore size is 0.25 μm to 10 μm, 0.25 μm to 5 μm, 0.25 μm to 2 μm, or 0.25 μm to 1 μm.

In some embodiments, the mean pore size is 0.5 μm to 1000 μm, 0.5 μm to 500 μm, 0.5 μm to 250 μm, 0.5 μm to 100 μm, 0.5 μm to 50 μm, 0.5 μm to 10 μm, 0.5 μm to 5 μm, 0.5 μm to 2 μm, or 0.5 μm to 1 μm.

In some embodiments, the mean pore size is 1 μm to 1000 μm, 1 μm to 500 μm, 1 μm to 250 μm, 1 μm to 100 μm, 1 μm to 50 μm, 1 μm to 25 μm, 1 μm to 10 μm, or 1 μm to 5 μm.

In some embodiments, the mean pore size is 2 μm to 1000 μm, 2 μm to 500 μm, 2 μm to 250 μm, 2 μm to 100 μm, 2 μm to 50 μm, 2 μm to 25 μm, 2 μm to 10 μm, or 2 μm to 5 μm.

In some embodiments, the mean pore size is 5 μm to 1000 μm, 5 μm to 500 μm, 5 μm to 250 μm, 5 μm to 100 μm, 5 μm to 50 μm, 5 μm to 25 μm, or 5 μm to 10 μm.

In some embodiments, the mean pore size is 10 μm to 1000 μm, 10 μm to 500 μm, 10 μm to 250 μm, 10 μm to 100 μm, 10 μm to 50 μm, or 10 μm to 25 μm.

In some embodiments, the mean pore size is 50 μm to 1000 μm, 50 μm to 500 μm, 50 μm to 250 μm, or 50 μm to 100 μm.

In some embodiments, the mean pore size is 100 μm to 1000 μm, 100 μm to 500 μm, or 100 μm to 250 μm.

In some embodiments, the mean pore size is 200 μm to 1000 μm, or 200 μm to 500 μm.

In some embodiments, matrices of the invention have a mean pore size (area) of at least 0.1 μm², at least 0.2 μm², at least 0.5 μm², at least 1 μm², at least 2 μm², at least 5 μm², at least 10 μm², at least 25 μm², at least 50 μm², at least 100 μm² or at least 200 μm².

In some embodiments, the mean pore size (area) is 1000 μm² or less, 750 μm² or less, 500 μm² or less, 250 μm² or less, 100 μm² or less, 50 μm² or less, 25 μm² or less, 10 μm² or less, 5 μm² or less, 2 μm² or less or 1 μm² or less.

In some embodiments, the mean pore size(area) is 0.1 μm² to 1000 μm², 0.1 μm² to 500 μm², 0.1 μm² to 250 μm², 0.1 μm² to 100 μm², 0.1 μm² to 50 μm², 0.1 μm² to 25 μm², 0.1 μm² to 10 μm², 0.1 μm² to 5 μm², 0.1 μm² to 1 μm² or 0.1 μm² to 1 μm².

In some embodiments, the mean pore size(area) is 0.25 μm² to 1000 μm², 0.25 μm² to 500 μm², 0.25 μm² to 250 μm², 0.25 μm² to 100 μm², 0.25 μm² to 50 μm², 0.25 μm² to 25 μm². In some embodiments, the mean pore size is 0.25 μm² to 10 μm², 0.25 μm² to 5 μm², 0.25 μm² to 2 μm² or 0.25 μm² to 1 μm².

In some embodiments, the mean pore size(area) is 0.5 μm² to 1000 μm², 0.5 μm² to 500 μm², 0.5 μm² to 250 μm², 0.5 μm² to 100 μm², 0.5 μm² to 50 μm², 0.5 μm² to 10 μm², 0.5 μm² to 5 μm², 0.5 μm² to 2 μm² or 0.5 μm² to 1 μm².

In some embodiments, the mean pore size(area) is 1 μm² to 1000 μm², 1 μm² to 500 μm²,1 μm² to 250 μm², 1 μm² to 100 μm², 1 μm² to 50 μm², 1 μm² to 25 μm², 1 μm² to 10 μm² or 1 μm² to 5 μm².

In some embodiments, the mean pore size(area) is 2 μm² to 1000 μm², 2 μm² to 500 μm², 2 μm² to 250 μm², 2 μm² to 100 μm², 2 μm² to 50 μm², 2 μm² to 25 μm², 2 μm² to 10 μm² or 2 μm² to 5 μm².

In some embodiments, the mean pore size(area) is 5 μm² to 1000 μm², 5 μm² to 500 μm², 5 μm² to 250 μm², 5 μm² to 100 μm², 5 μm² to 50 μm², 5 μm² to 25 μm² or 5 μm² to 10 μm².

In some embodiments, the mean pore size(area) is 10 μm² to 1000 μm², 10 μm² to 500 μm², 10 μm² to 250 μm², 10 μm² to 100 μm², 10 μm² to 50 μm² or 10 μm² to 25 μm².

In some embodiments, the mean pore size(area) is 50 μm² to 1000 μm², 50 μm² to 500 μm², 50 μm² to 250 μm² or 50 μm² to 100 μm².

In some embodiments, the mean pore size(area) is 100 μm² to 1000 μm², 100 μm² to 500 μm² or 100 μm² to 250 μm².

In some embodiments, the mean pore size(area) is 200 μm² to 1000 μm² or 200 μm² to 500 μm².

Optionally, with respect to the mean pore size ranges recited herein, less than 10% of the pores have a size below the lower end of the range and less than 10% of the pores have a size above the upper end of the range.

Optionally, with respect to the mean pore size ranges recited herein, less than 5% of the pores have a size below the lower end of the range and less than 5% of the pores have a size above the upper end of the range.

The matrix may have a porosity of at least 30% (0.3). The matrix may have a porosity of at least 40% (or 0.4). The matrix may have a porosity of at least 50% (0.5). The matrix may have a porosity of at least 80% (0.8). The matrix may have a porosity of at least 85% (0.85). The matrix may have a porosity of at least 90% (0.9). The matrix may have a porosity of at least 95% (0.95).

Matrices of the invention may comprise a polymer. The polymer may permit the effective formation of fibers. In some embodiments, the polymer may be a synthetic polymer. In some embodiments, the polymer is a natural polymer (such as collagen, chitosan or gelatin). In some embodiments, the polymer is selected from polyethylene oxide, polyvinyl alcohol and polyvinylpyrrolidone. Other polymers may include polycaprolactone or phosphino-carboxylic acid (PCA). A particularly preferred polymer is polycaprolactone (PCL). Other polymers may include poly(lactic-co-glycolic acid) (PLGA), polyurethane (PU), polylactic acid (PLA) or polyethylene oxide.

The polymer may be water soluble. The polymer may be soluble in an organic, or non-aqueous, solvent. The polymer may be soluble in a mixture of an aqueous and non-aqueous solvent. Suitable non-aqueous solvents may be, or may comprise, glycerol, dimethyl sulphoxide, ethylene glycol or propylene glycol. Suitable non-aqueous solvents may be acidic, for example, the acidic compound may comprise molecules with a carboxylic acid functional group. Other examples of non-aqueous solvents include acetic acid, acetone, chloroform, dimethylformamide, tetrahydrofuran and hexafluoroisopropanol.

Matrices of the invention may comprise two or more polymers. For example, PCL and collagen, PCL and PLGA, PLGA and chitosan, PVA and chitosan, PVA and gelatin or PU and gelatin.

In preferred embodiments the one or more fibers are biodegradable or bioasorbable.

In a preferred embodiment, the one or more fibers are electrospun fibers.

Matrices of the invention may thus be formed by electrospinning an electrospinnable composition. The electrospinnable composition may comprise an enzyme that is able to convert a substrate to release hydrogen peroxide and a substance that includes a substrate for the enzyme. According to the invention, there is provided an electrospinnable composition.

According to the invention, there is provided a method comprising electrospinning a composition comprising an enzyme that is able to convert a substrate to release hydrogen peroxide and a substance that includes a substrate for the enzyme. The electrospinnable composition may not comprise sufficient free water to allow the enzyme to convert the substrate. Examples of electrospinning hydrogen peroxide-generating compositions are provided in WO 2016/124926 and WO 2017/178822. For example, matrices of the invention may be produced by electrospinning compositions which comprise an enzyme that is able to convert a substrate to release hydrogen peroxide, a substance that includes a substrate for the enzyme and a polymer.

Electrospinnable compositions may comprise a non-aqueous solvent. Matrices of the invention may be produced by electrospinning compositions which comprise an enzyme that is able to convert a substrate to release hydrogen peroxide, a substance that includes a substrate for the enzyme, a polymer and a non-aqueous solvent. The non-aqueous solvent may be as described above. Following electrospinning, the matrix may be dried to evaporate the non-aqueous solvent. For example, the drying may be achieved by vacuum drying. Drying may occur for at least 12 hours, preferably at least 24 hours.

In one example, the matrix may be produced by electrospinning a composition comprising PCL and acetic acid.

The weight ratio of the polymer to substance+enzyme in the electrospinnable composition may be 10:1 or less (i.e. 10 parts polymer to 1 part substance+enzyme). The weight ratio may be from 10:1 to 1:1. The weight ratio may be 10:1 to 2:1.

The electrospinnable composition may comprise 0.1 g/ml to 0.2 g/ml of the polymer.

The electrospinnable composition may comprise 0.01 g/ml to 0.1 g/ml of the substance+enzyme.

In some embodiments, electrospinning is undertaken at a flow rate of 0.05 to 5 ml/min. In preferred embodiments, the flow rate is 0.05 to 0.15 ml/min. For example, the flow rate may be 0.1 ml/min.

In some embodiments, electrospinning is undertaken at an applied voltage of 5-50 kV. In some embodiments, the applied voltage is 10-20 kV. For example, the applied voltage may be 17 kV.

In some embodiments, electrospinning is undertaken at a collecting distance of 5-30 cm. In some embodiments, the collecting distance is 10-25 cm. For example, the collecting distance may be 18.7 cm.

The inventors have discovered that the fiber diameter and pore size of matrices of the invention can be manipulated by varying certain parameters. For example, the inventors have discovered that increasing the amount of the polymer relative to the substance+enzyme (e.g. SurgihoneyRO™) in the composition for electrospinning, may result in an increase in fiber diameter. In the field of electrospinning, it is known that larger pores can be achieved by increasing the diameter of electrospun fibers (J Rnjak-Kovacina et al. (2011)). Other techniques known in the art may also be used to manipulate pore size. For example, J. Wu et al. (2016) describes how pore size may also be manipulated by electrospinning with salt leaching, cryogenic electrospinning and electrospinning with sacrificial fibers.

As an alternative to electrospinning, matrices of the invention may be formed by coating pre-formed fibers with the enzyme and the substance that comprises a substrate for the enzyme. Coating may be achieved by a process such as electrospraying or by immersing in a composition comprising the enzyme and the substance that includes a substrate for the enzyme. Matrices of the invention may be produced by other methods, such as by 3D printing.

Although a fiber may be coated with the enzyme and substance, in certain embodiments, it is preferable that the enzyme and substance are integral with the fiber. The fiber may be said to be homogeneous, or the enzyme and substance may be substantially uniformly distributed within the fiber.

Matrices or compositions of the invention may comprise an unrefined substance that includes the substrate for the enzyme. The term “unrefined” is used herein to refer to substances that have not been processed into a pure form. Unrefined substances include substances that may have been concentrated, for example by drying or boiling. The substance may include one or more substrates from a natural source (termed herein a “natural substance”). Examples of natural substances include substances from a plant source, including from sap, roots, nectar, flowers, seeds, fruit, leaves, or shoots. The substance may be an unrefined natural substance, such as honey. The honey may be pasteurised. The honey may not contain catalase activity. The honey may not contain glucose oxidase. For example, the honey may have been heat-treated to inactivate any endogenous glucose oxidase and catalase. The honey may be creamed.

If the matrix or composition comprises an unrefined natural product, the enzyme is preferably additional to any enzyme activity which may already be present in the substance. In other words, the matrix or composition may comprise the substance and additional, or exogenous, enzyme.

Because honey is a natural product, its composition can vary greatly depending on its source. For example, the difference in antimicrobial potency among honeys can be more than one hundred-fold, depending on the geographical, seasonal and botanical source of the honey, as well as the harvesting, processing and storage conditions. Consequently, the antimicrobial efficacy may also vary depending on the type of honey used. Furthermore, honey may also contain other components, such as allergens e.g. trace amounts of pollen, which may cause adverse reactions when applied to certain subjects and make it unsuitable for certain pharmaceutical applications.

Honey may require processing such that it is in a suitable form for application to subjects, which can add cost and complexity to the production process. Such processing may include creaming or pasteurisation. Furthermore, for certain pharmaceutical applications, it may be difficult to obtain regulatory approval for honey-based compositions.

Although matrices and compositions based on natural products, such as honey, may be used in the invention, it may be desirable to provide matrices which provide some of the antimicrobial benefits provided by honey, but which also overcome some of its disadvantages.

So, the invention also concerns matrices that do not comprise an unrefined natural substance such as honey. For example, matrices or compositions of the invention may comprise a purified enzyme that is able to convert a substrate to release hydrogen peroxide and a substance that comprises a purified substrate for the enzyme (or a purified precursor substrate that can be converted to a substrate for the enzyme). Matrices of the invention may not comprise honey. Matrices of the invention may not comprise catalase. Matrices of the invention may not comprise phytochemicals.

Preferably, matrices of the invention, or the components of the matrices of the invention, are pharmaceutical grade.

The term “pharmaceutical grade” is used herein to include reference to a purity standard for a reagent that has been established by a recognized national or regional pharmacopeia (e.g., the U.S. Pharmacopeia (USP), British Pharmacopeia (BP), National Formulary (NF), European Pharmacopoeia (EP), or Japanese Pharmacopeia (JP)). Components of the matrix may be FDA-approved veterinary or human pharmaceutical substances.

Surprisingly, the applicant has found that compositions comprising a purified enzyme and a purified substrate or purified precursor-substrate can be more effective at killing microorganisms than known honey-based compositions that can generate hydrogen peroxide.

Preferably, the enzyme is an oxidoreductase enzyme. Examples of oxidoreductase enzymes that can convert a substrate to release hydrogen peroxide include glucose oxidase, hexose oxidase, cholesterol oxidase, galactose oxidase, pyranose oxidase, choline oxidase, pyruvate oxidase, glycollate oxidase, and amino acid oxidase. The corresponding substrates for these oxidoreductase enzymes are D-glucose, hexose, cholesterol, D-galactose, pyranose, choline, pyruvate, glycollate and amino acid, respectively.

A mixture of one or more oxidoreductase enzymes and one or more substrates for the oxidoreductase enzymes may be present in a composition of the invention.

According to a preferred embodiment of the invention, the oxidoreductase enzyme is glucose oxidase and the substrate is D-glucose.

References herein to “enzyme” refer to one or more enzyme. For example, in some embodiments, compositions of the invention may comprise a plurality of enzymes that are able to convert a substrate to release hydrogen peroxide. In some embodiments, compositions of the invention may comprise only one enzyme that is able to convert a substrate to release hydrogen peroxide.

The term “purified enzyme” is used herein to include an enzyme preparation in which the enzyme has been separated from at least some of the impurities originally present when the enzyme was produced. Preferably, impurities that have been removed or reduced include those that would otherwise interfere with the ability of the enzyme to convert the substrate to release hydrogen peroxide.

It may not always be necessary or desirable that the purified enzyme is at a high level of purity provided that the enzyme is able to convert the substrate to release hydrogen peroxide. In some circumstances, it may be desirable to use a relatively crude enzyme preparation. Examples of suitable purity levels include at least 10%, 20%, 30%, 40%, 50%, 60%, 70%, 80%, or 90% pure (mass purity). Preferably, the enzyme is at least 90% pure. More preferably, the enzyme is at least 95% pure. Even more preferably, the enzyme is at least 99% pure.

The enzyme may have been produced by recombinant or non-recombinant means, and may be a recombinant or non-recombinant enzyme. The enzyme may be purified from a microbial source.

If the enzyme is glucose oxidase, it may be a purified natural glucose oxidase preparation. The activity of the glucose oxidase may be selected depending on the desired rate of production of hydrogen peroxide following dilution of the storage-stable composition. Several glucose oxidase preparations are commercially available (glucose oxidase is identified by the reference CAS:9001-37-0). Common microbial sources for glucose oxidase from non genetically modified organisms include selected strains of Aspergillus niger, Penicillium amagasakiense, Penicillium variabile, Penicillium notatum. Medical device grade glucose oxidase, from GMO Aspergillus niger, is available from Biozyme UK, activity 240 iu/mg. Food standard glucose oxidase, from Aspergillus niger, is available from BIO-CAT INC, activity 15,000 Units/g. Non-Genetically Modified glucose oxidase is available from BIO-CAT INC, activity 12,000/g. Glucose oxidase (G03B2), from Apsergillus niger, is available from BBI Enzymes Limited, activity 360 Units/mg. Contaminants: alpha amylase no greater than 0.05%, Saccharase no greater than 0.05%, maltase no greater than 0.05% and GO/Cat no less than 2000.

The level of purity of the enzyme may be selected as appropriate depending on the intended use of the composition. For medical use, a medical grade or medical device grade of purity may be used. For pharmaceutical use, a pharmaceutical grade of purity may be used.

Matrices or compositions (such as electrospinnable compositions) of the invention may be produced by initially adding purified enzyme to a substance that comprises the substrate. For example, purified enzyme may be added to honey, or purified enzyme may be added to a purified substrate (see below). According to the invention, there is provided a method comprising adding purified enzyme to a substance comprising a substrate (preferably a purified, substrate) for the enzyme, and then forming the matrix. The matrix may be formed by electrospinning, as described herein.

Matrices or compositions of the invention may comprise sufficient enzyme and substrate to provide for sustained release of hydrogen peroxide at a specific level or concentration.

Matrices or compositions of the invention may comprise sufficient enzyme and substrate to provide for sustained release of hydrogen peroxide at a level of less than 2 mmol/litre for a period of at least twenty four hours, following dilution of the composition.

Matrices or compositions of the invention may comprise sufficient enzyme and substrate to provide for sustained release of at least 0.02, 0.03, 0.04, 0.05, 0.1, 0.2, 0.3, 0.4, 0.5, 1 or 1.5 mmol/litre hydrogen peroxide for a period of at least 24 hours, more preferably 48 hours.

So, in some embodiments, matrices or compositions of the invention may comprise sufficient enzyme and substrate to provide for sustained release of 0.1 to 2 mmol/litre hydrogen peroxide for a period of at least 24 hours, more preferably 48 hours.

For example, in some embodiments, compositions of the invention may provide for sustained release of hydrogen peroxide at a concentration of at least 2 ppm, at least 5 ppm, at least 10 ppm, at least 20 ppm or at least 50 ppm. In preferred embodiments, the level may be at least 2 ppm. In some embodiments, the concentration may be, at the most, 500 ppm, 200 ppm, 100 ppm, 50 ppm, 20 ppm or 10 ppm. In preferred embodiments, the level may be 20 ppm or less. In even more preferred embodiments, the level may be 10 ppm or less. For example, the concentration may be 10 to 500 ppm, 20 to 200 ppm or 50 to 100 ppm, 2 to 50 ppm, 2 to 20 ppm or 5 to 10 ppm. If the matrix or composition does not comprise sufficient free water to allow the enzyme to convert the substrate, hydrogen peroxide production may only occur once it has been diluted by water and there is sufficient free water to allow the enzyme to convert the substrate. Addition of water may thus initiate hydrogen peroxide production. Matrices or compositions, of the invention may provide for sustained release of hydrogen peroxide for at least 1 hour, at least 12 hours, at least 24 hours, at least 2 days, or at least 4 days. Preferably, the level of hydrogen peroxide is sustained for at least 4 days. In preferred embodiments, the level of hydrogen peroxide is sustained at 10 to 500 ppm for at least 1 hour, at least 12 hours, at least 24 hours, at least 2 days, or at least 4 days. In other embodiments, the level of hydrogen peroxide is sustained at 50 to 100 ppm for at least 1 hour, at least 12 hours, at least 24 hours, at least 2 days, or at least 4 days. In other embodiments, the level of hydrogen peroxide is sustained at 2 to 50 ppm for at least 12 hours, at least 24 hours, at least 2 days, or at least 4 days. In other embodiments, the level of hydrogen peroxide is sustained at 5 to 10 ppm for at least 12 hours, at least 24 hours, at least 2 days, or at least 4 days. In some embodiments, matrices or compositions of the invention may provide for sustained release of 2 to 500 ppm hydrogen peroxide for at least 24 hours.

A matrix of the invention may provide for release of hydrogen peroxide at a concentration of at least 150 μM, optionally at one hour, or 24 hours, following a 1:1 (by weight) dilution of the matrix with water.

A matrix of the invention may provide for release of hydrogen peroxide at a concentration of at least 250 μM, optionally at one hour, or 24 hours, following a 1:1 (by weight) dilution of the matrix with water.

A matrix of the invention may provide for release of hydrogen peroxide at a concentration of at least 500 μM, optionally at one hour, or 24 hours, following a 1:1 (by weight) dilution of the matrix with water.

A matrix of the invention may provide for release of hydrogen peroxide at a concentration of at least 1,000 μM, optionally at one hour, or 24 hours, following a 1:1 (by weight) dilution of the matrix with water.

A matrix of the invention may provide for release of hydrogen peroxide at a concentration of at least 1,500 μM, optionally at one hour, or 24 hours, following a 1:1 (by weight) dilution of the matrix with water.

A matrix of the invention may provide for release of hydrogen peroxide at a concentration of at least 2,000 μM, optionally at one hour, or 24 hours, following a 1:1 (by weight) dilution of the matrix with water.

A matrix of the invention may provide for release of hydrogen peroxide at a concentration of at least 5,000 μM, optionally at one hour, or 24 hours, following a 1:1 (by weight) dilution of the matrix with water.

A matrix of the invention may provide for release of hydrogen peroxide at a concentration of at least 10,000 μM, optionally at one hour, or 24 hours, following a 1:1 (by weight) dilution of the matrix with water.

A matrix of the invention may provide for release of hydrogen peroxide at a concentration of 5,000 μM or less, optionally at one hour, or 24 hours, following a 1:1 (by weight) dilution of the matrix with water.

A matrix of the invention may provide for release of hydrogen peroxide at a concentration of 2,000 μM or less, optionally at one hour, or 24 hours, following a 1:1 (by weight) dilution of the matrix with water.

A matrix of the invention may provide for release of hydrogen peroxide at a concentration of 1,000 μM or less, optionally at one hour, or 24 hours, following a 1:1 (by weight) dilution of the matrix with water.

A matrix of the invention may provide for release of hydrogen peroxide at a concentration of matrix with water.

A matrix of the invention may provide for release of hydrogen peroxide at a concentration of 10,000 μM or less, optionally at one hour, or 24 hours, following a 1:1 (by weight) dilution of the matrix with water.

A matrix of the invention may provide for release of hydrogen peroxide at a concentration of 20,000 μM or less, optionally at one hour, or 24 hours, following a 1:1 (by weight) dilution of the matrix with water.

A matrix of the invention may provide for release of hydrogen peroxide at a concentration of 30,000 μM or less, optionally at one hour, or 24 hours, following a 1:1 (by weight) dilution of the matrix with water.

A matrix of the invention may provide for release of hydrogen peroxide at a concentration of 50,000 μM or less, optionally at one hour, or 24 hours, following a 1:1 (by weight) dilution of the matrix with water.

A matrix of the invention may provide for release of hydrogen peroxide at a concentration of 80,000 μM or less, optionally at one hour, or 24 hours, following a 1:1 (by weight) dilution of the matrix with water.

A matrix of the invention may provide for release of hydrogen peroxide at a concentration of 100,000 μM or less, optionally at one hour, or 24 hours, following a 1:1 (by weight) dilution of the matrix with water.

A matrix of the invention may provide for release of hydrogen peroxide at a concentration of 500 to 100,000 μM, optionally at one hour, or 24 hours, following a 1:1 (by weight) dilution of the matrix with water.

A matrix of the invention may provide for release of hydrogen peroxide at a concentration of 500 to 50,000 μM, optionally at one hour, or 24 hours, following a 1:1 (by weight) dilution of the matrix with water.

A matrix of the invention may provide for release of hydrogen peroxide at a concentration of 500 to 10,000 μM, optionally at one hour, or 24 hours, following a 1:1 (by weight) dilution of the matrix with water.

A matrix of the invention may provide for release of hydrogen peroxide at a concentration of 500 to 5,000 μM, optionally at one hour, or 24 hours, following a 1:1 (by weight) dilution of the matrix with water.

A matrix of the invention may provide for release of hydrogen peroxide at a concentration of 500 to 2,500 μM, optionally at one hour, or 24 hours, following a 1:1 (by weight) dilution of the matrix with water.

A matrix of the invention may provide for release of hydrogen peroxide at a concentration of 500 to 2,000 μM, optionally at one hour, or 24 hours, following a 1:1 (by weight) dilution of the matrix with water.

A matrix of the invention may provide for release of hydrogen peroxide at a concentration of 150 to 2,000 μM, optionally at one hour, or 24 hours, following a 1:1 (by weight) dilution of the matrix with water.

A matrix of the invention may provide for release of hydrogen peroxide at a concentration of 150 to 1,000 μM, optionally at one hour, or 24 hours, following a 1:1 (by weight) dilution of the matrix with water.

A matrix of the invention may provide for release of hydrogen peroxide at a concentration of 150 to 500 μM, optionally at one hour, or 24 hours, following a 1:1 (by weight) dilution of the matrix with water.

The level of hydrogen peroxide produced on dilution of matrices of the invention may be sustained at particular levels for a period of time. For example, the level of hydrogen peroxide may be 200 μM to 5,000 μM, preferably at least 400 μM to 2,000 μM for at least 72 hours following dilution of the matrix e.g. following a 1:1 (by weight) dilution of the matrix with water.

Matrices of the invention may comprise sufficient enzyme and substrate to provide for sustained release of hydrogen peroxide at a level of less than 3 mmol/litre for a period of at least twenty four hours following dilution of the matrix, e.g. following a 1:1 (by weight) dilution of the matrix with water.

Matrices of the invention may comprise sufficient enzyme and substrate to provide for sustained release of hydrogen peroxide at a level of less than 2 mmol/litre for a period of at least twenty four hours following dilution of the matrix, e.g. following a 1:1 (by weight) dilution of the matrix with water.

Matrices of the invention may comprise sufficient enzyme and substrate to provide for sustained release of at least 0.2, 0.3, 0.4, 0.5, 1 or 1.5 mmol/litre hydrogen peroxide for a period of at least 24 hours, more preferably 48 hours, optionally following dilution of the matrix, e.g. following a 1:1 (by weight) dilution of the composition with water.

So, in some embodiments, matrices of the invention may comprise sufficient enzyme and substrate to provide for sustained release of 0.2 to 3 mmol/litre hydrogen peroxide for a period of at least 24 hours, more preferably 48 hours, e.g. following a 1:1 (by weight) dilution of the matrix with water.

In some embodiments, matrices of the invention may comprise sufficient enzyme and substrate to provide for sustained release of 0.3 to 2 mmol/litre hydrogen peroxide for a period of at least 24 hours, more preferably 48 hours, e.g. following a 1:1 (by weight) dilution of the matrix with water.

Levels of hydrogen peroxide may be quantified following the method of Kerkvliet 1996 and Serrano et al., 2004, using Merckoquant test strip (no. 10011; Merck, Germany). Matrices or compositions of the invention may comprise 10 to 2000 ppm of the enzyme. Matrices or compositions of the invention may comprise 25 to 2000 ppm of the enzyme, for example 50 to 1000 ppm of the enzyme. Matrices or compositions of the invention may comprise 750 to 2000 ppm of the enzyme. Matrices or compositions of the invention may comprise 250 to 1500 of the enzyme.

Matrices or compositions of the invention may comprise at least 10 ppm of the enzyme. Matrices compositions of the invention may comprise at least 25 ppm of the enzyme. Matrices or compositions of the invention may comprise at least 50 ppm of the enzyme. Matrices or compositions of the invention may comprise at least 100 ppm of the enzyme. Matrices or compositions of the invention may comprise at least 250 ppm of the enzyme. Matrices or compositions of the invention may comprise at least 500 ppm of the enzyme. Matrices or compositions of the invention may comprise at least 1000 ppm of the enzyme.

Matrices or compositions of the invention may comprise 2000 ppm or less of the enzyme. Matrices or compositions of the invention may comprise 1500 ppm or less of the enzyme, Matrices or compositions of the invention may comprise 1000 ppm or less of the enzyme.

Matrices or compositions of the invention may comprise the enzyme in an amount of 0.001% by weight to 0.05% by weight. Preferably, compositions of the invention may comprise the enzyme in an amount of 0.005% by weight to 0.02% by weight.

The enzyme activity (for example, the glucose oxidase activity) may range, for example, from 1-400 IU/mg, or 1-300 IU/mg, for example 250-280 IU/mg. The amount of enzyme used is likely to depend on several factors, including the desired use of the composition, the desired level of hydrogen peroxide release, and the desired length of time for hydrogen peroxide release. A suitable amount of enzyme can readily be determined by a person of ordinary skill in the art, if necessary using a well diffusion assay, to determine the extent of hydrogen peroxide release for different amounts of enzyme. Suitable amounts of enzyme (such as glucose oxidase) may be from 0.0001% to 0.5% w/w of the composition. The amount of enzyme used may be selected so as to produce a composition for generating antimicrobial activity that is equivalent to a selected phenol standard (for example a 10%, 20%, or 30% phenol standard).

Matrices or compositions of the invention may comprise at least 1 unit, and preferably up to 1500 units, of the enzyme per gram of the composition. A “unit” is defined herein as the amount of enzyme (e.g. glucose oxidase) causing the oxidation of 1 micromole of substrate (e.g. glucose) per minute at 25 degrees centigrade at pH 7.0.

In some embodiments, a matrix or composition according to the invention comprises more than 15 units, for example at least 30 units, at least 50 units, or at least 100 units, and suitably less than 685 units, for example 100-500 units, of enzyme (e.g. glucose oxidase) per gram of the composition.

In other embodiments, a matrix or composition of the invention comprises at least 500 units, for example 500-1000 units, or 685-1000 units, of enzyme (e.g. glucose oxidase) per gram of the composition.

References herein to “substrate” or “precursor-substrate” refer to one or more substrate or precursor-substrate. For example, in some embodiments, matrices or compositions of the invention may comprise a plurality of substrates or precursor-substrates. In some embodiments, matrices or compositions of the invention may comprise only one substrate or only one precursor substrate.

The term “purified substrate” or “purified precursor-substrate” is used herein to include a substrate or precursor-substrate preparation in which the substrate or precursor-substrate has been separated from at least some of the impurities originally present when the substrate or precursor-substrate was obtained or produced. The purified substrate or precursor-substrate may be obtained from a natural source or may be synthetically produced. The purified substrate or precursor-substrate may be a processed, extracted, or refined substrate or precursor-substrate (i.e. a substrate or precursor-substrate in which impurities or unwanted elements have been removed by processing).

It may not always be necessary or desirable that the purified substrate or precursor substrate is at a high level of purity provided that the enzyme is able to convert the substrate to release hydrogen peroxide. In some circumstances, it may be desirable to use a relatively crude substrate or precursor-substrate preparation. Examples of suitable purity levels include at least 10%, 20%, 30%, 40%, 50%, 60%, 70%, 80%, 90%, 95%, or 99% pure (mass purity). Preferably the purity level is at least 90%, more preferably at least 95%, even more preferably at least 99%. However, in some embodiments, it may be desirable that the purified substrate or purified precursor-substrate is a medical grade, medical device grade, or pharmaceutical grade substrate or precursor-substrate.

The substance may be or may comprise a sugar substance. In particular embodiments, the purified substrate or precursor substrate is or comprises a purified sugar. The term “sugar” is used herein to refer to a carbohydrate with the general formula Cₘ(H₂O)ₙ. The purified sugar may be obtained from a natural source (for example a processed, extracted, or refined natural sugar), or be synthetically produced. The purified sugar may be at least 10%, 20%, 30%, 40%, 50%, 60%, 70%, 80%, 90%, 95%, or 99% pure (mass purity). Preferably, the purity level is at least 90%. Even more preferably, the purity level is at least 99%. The purified sugar may be a medical grade, medical device grade, or pharmaceutical grade sugar. The sugar may include, for example purified D-glucose, hexose, or D-galactose. For example, the purified sugar may be medical grade, medical device grade, or pharmaceutical grade D-glucose, hexose, or D-galactose.

In particular embodiments, the enzyme and the substrate are purified, for example purified glucose oxidase and purified D-glucose, suitably medical grade, medical device grade, or pharmaceutical grade glucose oxidase and D-glucose.

In some embodiments, the enzyme, the substrate (or precursor substrate), and optionally the solute are each at least 95% pure.

In some embodiments, the enzyme, the substrate (or precursor substrate), and optionally the solute are each at least 99% pure.

For compositions of the invention which comprise a precursor-substrate, the composition may comprise one or more enzymes (preferably purified enzymes) for converting the precursor-substrate to the substrate for the enzyme. However, in some embodiments, the precursor-substrate may not necessarily be converted to the substrate enzymatically. For example, for some precursor substrates, addition of water may be sufficient for conversion. Alternatively or additionally, compositions of the invention may comprise non-enzymatic catalysts.

Matrices or compositions of the invention which comprise a precursor-substrate may comprise a first enzyme that is able to convert the substrate to release hydrogen peroxide, and a second enzyme that is able to convert the precursor-substrate to the substrate for the first enzyme.

The precursor-substrate is preferably a carbohydrate, such as a polysaccharide, or a sugar e.g. a disaccharide, or sugar derivative.

For example, the precursor-substrate may be sucrose, the first enzyme may be glucose oxidase and the second enzyme may be invertase.

In another example, the precursor-substrate may be maltose, the first enzyme may be glucose oxidase and the second enzyme may be maltase.

Compositions of the invention which comprise a precursor-substrate may comprise an enzyme (preferably a purified enzyme) that is able to convert the substrate to release hydrogen peroxide, and at least two enzymes (e.g. second and third enzymes, preferably purified enzymes) that are able to convert the precursor-substrate to the substrate for the first enzyme.

For example, the precursor-substrate may be starch, the first enzyme may be glucose oxidase and the second and third enzymes may be amylase and maltase.

For example, the precursor-substrate may be cellulose, the first enzyme may be glucose oxidase and the second and third enzymes may be cellulose and beta-glucosidase.

In some embodiments, matrices or compositions of the invention may comprise both a substrate that can be converted by the enzyme to generate hydrogen peroxide, and a precursor-substrate that can be converted to the substrate. In some embodiments, matrices or compositions of the invention may have the precursor substrate as an alternative to the substrate.

Matrices or compositions of the invention may comprise an additional component which is preferably a solute. References herein to “solute” refer to one or more solute. For example, in some embodiments, matrices or compositions of the invention may comprise a plurality of solutes. In some embodiments, the composition may only comprise one solute. Preferably the solute is soluble in water.

The solute may be distinct from the substrate, or in some examples, the substrate may be same as the solute. For example, the composition may comprise fructose and fructose oxidase: the fructose being both the solute and the substrate for enzyme. In another example, the substrate may be glucose and the solute may be fructose.

The solute is preferably purified, meaning that the solute has been separated from at least some of the impurities originally present when the solute was obtained or produced. The purified solute may be obtained from a natural source or may be synthetically produced. The purified solute may be a processed, extracted, or refined substrate (i.e. a solute in which impurities or unwanted elements have been removed by processing).

It may not always be necessary or desirable that the purified solute is at a high level of purity. In some circumstances, it may be desirable to use a relatively crude solute preparation. Examples of suitable purity levels include at least 10%, 20%, 30%, 40%, 50%, 60%, 70%, 80%, 90%, 95%, or 99% pure (mass purity). Preferably, the purity level is at least 90%. More preferably, the purity level is at least 95%. Even more preferably, the purity level is at least 99%. In some embodiments, it may be desirable that the solute is a medical grade, medical device grade, or pharmaceutical grade solute.

The solute may be a carbohydrate. The solute may be a polysaccharide. Preferably, the solute is a sugar or sugar derivative. More preferably, the solute is a sugar. Suitable sugars include oligosaccharides, disaccharides or monosaccharides. Preferably, the sugar is a disaccharide or a monosaccharide. In particularly preferred embodiments, the sugar is a monosaccharide. Suitable sugars may include fructose, glucose, galactose, sucrose and maltose. In a particularly preferred embodiment, the sugar is fructose.

The term “sugar derivative” is used herein to refer to a sugar that has been modified by addition of one or more substituents other than a hydroxyl group. Sugar derivatives, thus encompass amino sugars, acidic sugars, deoxy sugars, sugar alcohols, glycosylamines and sugar phosphates. For example, sugar derivatives may include glucose-6-phosphateglucosamine, glucoronate, gluconate, galactosamine, glucosamine, sialic acid, deoxyribosefucose, rhamnose glucuronic acid, polyols (e.g. sorbitol, erythritol, xylitol, mannitol, lactitol and maltitol) and sucralose.

Matrices or compositions of the invention may comprise two or more solutes, as described herein. For example, compositions of the invention may comprise two or more sugars or sugar derivatives. The composition may comprise a maximum of two solutes, e.g. two sugars or sugar derivatives; or a maximum of three solutes, e.g. three sugars or sugar derivatives. For instance, a composition of the invention may comprise glucose, fructose and sucrose.

The solute preferably has a high solubility in water, for example a solubility which is greater than glucose. Glucose has a solubility of 90 g/100 g water at 20° C. and 1 atm. In a preferred embodiment, the solute has a solubility greater than or equal to 100 g/100 g water at 20° C. and 1 atm. In a more preferred embodiment, the solute has a solubility greater than or equal to 200 g/100 g water at 20° C. and 1 atm. In an even more preferred embodiment, the solute has a solubility greater than or equal to 300 g/100 g water at 20° C. and 1 atm.

A solute with a high solubility may be advantageous because if the composition of the invention is a solution, it may enable the solution to have a high concentration of solutes, which may in turn provide a high osmolarity or osmotic strength. Compositions with a high osmolarity or osmotic strength may assist with the antimicrobial efficacy of the composition because they may reduce the amount of water available for microbes or draw water away from microbes, and may assist in wound healing and wound debridement.

Fructose is a particularly preferred solute because it has a solubility of about 375 g/100 g water at 20° C. and 1 atm. Consequently, the solute may be fructose.

In some embodiments, the solute with a solubility of at least 100 g/100 g water at 20° C. and 1 atm, at least 200 g/100 g water at 20° C. and 1 atm or at least 300 g/100 g water at 20° C. and 1 atm, may be the purified substrate. So, for example, a matrix or composition of the invention may comprise fructose and fructose oxidase.

Matrices or compositions of the invention may thus comprise only one sugar or sugar derivative which is the solute and the substrate, and one enzyme for converting the substrate and generating hydrogen peroxide.

In some embodiments, the solute with the solubility of at least 100 g/100 g water at 20° C. and 1 atm, at least 200 g/100 g water at 20° C. and 1 atm or at least 300 g/100 g water at 20° C. and 1 atm, may be distinct from the purified substrate. For example, a matrix or composition of the invention may comprise glucose, glucose oxidase and fructose.

In preferred embodiments, the purified substrate is a sugar or sugar derivative (e.g. glucose) and the solute is a sugar or sugar derivative (e.g. fructose).

Preferably, the matrix or composition comprises at least two sugars or sugar derivatives (e.g. including glucose and fructose). The composition may comprise a maximum of two sugars or sugar derivatives (e.g. only glucose and fructose).

The enzyme, purified substrate (or precursor substrate) and purified solute may be referred to as a “synthetic honey”. For example, a synthetic honey may comprise glucose oxidase, glucose and fructose. So, in matrices or compositions of the invention, the substance that includes a substrate for the enzyme may be, or may comprise, the substrate and the solute.

A honey or synthetic honey may be combined with a polymer and a non-aqueous solvent to form an electrospinnable composition of the invention, which in turn is used to form a matrix of the invention.

According to the invention, there is provided a matrix comprising a purified enzyme that is able to convert a substrate to release hydrogen peroxide and a substance, the substance including a purified substrate for the enzyme (and/or a purified precursor-substrate that can be converted to a substrate for the enzyme) and a purified solute.

According to the invention, there is provided a matrix comprising an enzyme (preferably a purified enzyme) that is able to convert a substrate to release hydrogen peroxide and a substance, the substance including a purified substrate for the enzyme (and/or a purified precursor-substrate that can be converted to a substrate for the enzyme) and a purified solute, wherein the solute has a solubility of at least 100 g/100 g water at 20° C. and 1 atm.

According to the invention, there is provided a method comprising electrospinning a composition comprising an enzyme (preferably a purified enzyme) that is able to convert a substrate to release hydrogen peroxide, a substance, a polymer and a non-aqueous solvent, wherein the substance includes a purified substrate for the enzyme (and/or a purified precursor-substrate that can be converted to a substrate for the enzyme) and a purified solute.

According to the invention, there is provided a method comprising electrospinning a composition comprising an enzyme (preferably a purified enzyme) that is able to convert a substrate to release hydrogen peroxide, a substance, a polymer and a non-aqueous solvent, wherein the substance includes a purified substrate for the enzyme and a purified solute, wherein the solute has a solubility of at least 100 g/100 g water at 20° C. and 1 atm.

The synthetic honey may have properties similar to naturally-occurring honey. The honey or synthetic honey may have a viscosity, such as a dynamic viscosity, of at least 5000 mPas at 20° C., more preferably at least 7500 at 20° C. The honey or synthetic honey may have a viscosity of 5000 to 20000 mPas at 20° C., more preferably 7500 to 12000 mPas at 20° C.

Substances for use in the invention may comprise at least 5% by weight of sugars and/or sugar derivatives. Substances for use in the invention may comprise at least 10% by weight of sugars and/or sugar derivatives. Substances for use in the invention may comprise at least 25% by weight of sugars and/or sugar derivatives. Substances for use in the invention may comprise at least 50% by weight of sugars and/or sugar derivatives. Substances for use in the invention may comprise 95% by weight or less of sugars or sugar derivatives. Substances for use in the invention may comprise at 75% by weight or less of sugars and/or sugar derivatives. For example, substances for use in of the invention may comprise 10% to 95% by weight sugars and/or sugar derivatives. Substances for use in the invention may comprise 25% to 75% by weight sugars and/or sugar derivatives. Substances for use in the invention may comprise 50 to 95% by weight sugars and/or sugar derivatives.

Substances for use in the invention may comprise 5 to 50% by weight of substrate for the enzyme (e.g. glucose) or 5 to 50% by weight of the precursor substrate that can be converted to the substrate for the enzyme. For instance, substances for use in the invention may comprise 5 to 25% by weight of substrate for the enzyme (e.g. glucose) or 5 to 25% by weight of the precursor substrate that can be converted to the substrate for the enzyme

Substances for use in the invention which are liquids or solutions may comprise at least 70%, by weight of substrate and solute (e.g. wherein the solute is a sugar or derivative), more preferably at least 75%, by weight of substrate and solute (e.g. wherein the solute is a sugar or derivative), and even more preferably, at least 80% by weight of substrate and solute(e.g. wherein the solute is a sugar or derivative). For example, where the substance comprises glucose and fructose, the glucose and fructose may be present in a total amount of at least 80%, by weight. The solute may be present in an amount of at least 40%, preferably at least 50%. The purified substrate (e.g. glucose) may be present in an amount of at least 20% by weight, preferably at least 25% by weight, more preferably at least 30% by weight. So, in one example, a solute (e.g. fructose) is present in an amount of 40 to 60% by weight and a substrate (e.g. glucose) is present in an amount of 20 to 40% by weight. In another example, a substrate (e.g. glucose) is present in an amount of 25 to 35% by weight and a solute (e.g. fructose) is present in an amount of 45 to 55%, by weight.

Substances for use in the invention which are liquids or solutions may comprise at least 70%, by weight sugar or sugar derivative, more preferably at least 75%, by weight sugar or sugar derivative, and even more preferably, at least 80%, by weight, sugar or sugar derivative. For example, in a preferred embodiment, the substance comprises glucose and fructose. Preferably, the glucose and fructose is present in an amount of at least 80% by weight of the composition.

In substances for use in the invention that are solutions or liquids, water may be present in an amount which is less than 20% by weight, but preferably greater than 10% by weight, more preferably greater than 15%, by weight. For example, water may be present in an amount between 10 and 20%, by weight, or in an amount of 10 to 20% by weight.

Substances for use in the invention may comprise at least 90% by dry weight of the substrate and the solute (preferably a sugar or sugar derivative), combined. Substances for use in the invention may comprise at least 95% by dry weight of the substrate and the solute (preferably a sugar or sugar derivative), combined.

Substances for use in the invention may comprise at least 90% dry weight of sugar or sugar derivative. Compositions of the invention may comprise at least 95% by dry weight of sugar or sugar derivative.

Substances for use in the invention may comprise at least 60%, dry weight of the solute (e.g. a sugar or sugar derivative). The substrate may be at least 30%, dry weight of the composition.

Substances for use in the invention may comprise 5 to 75% by weight of solute (e.g. fructose). For instance, substances for use in the invention may comprise 10 to 50% by weight of solute.

Matrices or compositions of the invention, or substances for use in the invention, may comprise a buffer, or a component that may be capable of acting as a buffer in an aqueous solution. An example of a suitable buffer is a citric acid/NaOH buffer, such as a 50 mMol citric acid/NaOH buffer. Matrices or compositions of the invention, or substances for use in the invention, may be buffered (or may be capable of being buffered in an aqueous solution) at a pH of 5 or less, e.g. 3 to 5 (such as about pH 4). Alternatively, matrices or compositions of the invention, or substances for use in the invention, may be buffered (or may be capable of being buffered in an aqueous solution) at a pH greater than 5, e.g. 6 to 8 (such as about pH 7). The pH may be 3.5 to 6, 4.5 to 5.5, or 5.0 to 7.5.

Matrices or compositions of the invention may have no added peroxidase. Matrices or compositions of the invention may comprise substantially no peroxidase. Matrices or compositions of the invention may be essentially free of peroxidase.

Matrices or compositions of the invention may have no added zinc oxide. Matrices or compositions of the invention may comprise substantially no zinc oxide. Matrices or compositions of the invention may be essentially free of zinc oxide.

Matrices of the invention may be sterile. The matrices may be sterilised by any suitable means. Preferably, matrices of the invention have been sterilised by irradiation, such as gamma irradiation or electron beam irradiation. A suitable level of gamma irradiation is 10-70 kGy, preferably 25-70 kGy, more preferably 35-70 kGy. A suitable level or dose of irradiation (e.g. electron beam irradiation) may be 10-100 kGy, preferably 30-80 kGy, more preferably 50-80kGy. The dose may be greater than 35 kGy. The dose may be less than 80 kGy, for example 75 kGy or less. In one embodiment, compositions of the invention may be sterilised by irradiation that is not gamma irradiation.

The matrix may be sterilised by a method other than exposure to irradiation. For example, the matrix may be sterilised by treatment with ethanol.

There is also provided according to the invention a method of sterilising a matrix of the invention, which comprises exposing the matrix to irradiation, preferably gamma irradiation or electron beam irradiation.

Since ozone has not been authorised by the US FDA for sterilisation of honey-based products for use in wound healing, matrices according to the invention preferably have not been sterilized by ozonation, and do not include ozone, or any components that have been subjected to sterilisation by ozonation. In particular, matrices according to the invention should not comprise ozonized honey or ozonated oil.

The matrix may be placed in sealed packaging. This may to help maintain sterility. The packaging is preferably opaque.

In preferred embodiments, the matrix is bioabsorbable or biodegradable.

A matrix according to the invention may be impregnated or seeded with one or more tissue cells. The one or more cells may be mammalian cells, such as human cells. The one or more tissue cells may comprise a skin cell. The one or more tissue cells may comprise a stem cell. The stem cell may be a pluripotent stem cell (such as an induced pluripotent stem cell) or a multipotent stem cell. The stem cell is preferably an adult or somatic stem cell. The one or more tissue cells may comprise a human adipose-derived stem cell (hADSC).

The matrix of the invention may be used for tissue regeneration, for tissue engineering or for repairing damaged tissue. The damaged tissue may be damaged skin. The matrix may be applied or administered to the damaged tissue. Preferably, once the matrix has been applied or administered to the damaged tissue, it is preferably not manually removed or replaced because removal or replacement may damage the tissue that has been forming within and around the matrix. Once the matrix has assisted in tissue repair, it may be absorbed into the living tissue.

The matrix may be implanted into a subject's tissue, for example into a subject's damaged tissue.

The matrix may be administered to the damaged tissue without being impregnated or seeded with cells. Consequently, the matrix may provide support for the attachment and proliferation of a subject's own cells at the site of tissue damage. Alternatively, the matrix may be impregnated or seeded with cells prior to contacting with the damaged tissue. The cells may be the subject's own cells (i.e. endogenous cells), or they may be exogenous cells. The cells may be stem cells.

According to the invention, there is provided a method of proliferating cells comprising contacting cells with a matrix of the invention. The cells may be contacted with the matrix in vivo, ex vivo or in vitro. The cells and the matrix may be incubated in a nutrient medium following contacting the cells with the matrix. The method may comprise contacting cells with the matrix and a nutrient medium. The method may comprise incubation.

According to the invention, there is provided a method of differentiating cells comprising contacting cells with a matrix of the invention. The cells may be contacted with the matrix in vivo, ex vivo or in vitro. The cells and the matrix may be incubated in a nutrient medium following contacting the cells with the matrix. The method may comprise contacting cells with the matrix and a nutrient medium. The method may comprise incubation.

According to the invention there is provided tissue or cell culture comprising a matrix of the invention.

According to the invention, there is provided a method of repairing damaged tissue comprising administering a matrix of the invention, to damaged tissue.

According to the invention, there is provided a matrix of the invention for use in repairing damaged tissue.

According to the invention, there is provided a use of a matrix of the invention in the manufacture of a medicament for repairing damaged tissue.

According to the invention, there is provided use of a matrix as a tissue scaffold, the matrix comprising an enzyme that is able to convert a substrate to release hydrogen peroxide and a substance that includes a substrate for the enzyme.

According to the invention, there is provided use of a matrix in the manufacture of a medicament for supporting attachment, proliferation and/or differentiation of cells, the matrix comprising an enzyme that is able to convert a substrate to release hydrogen peroxide and a substance that includes a substrate for the enzyme.

According to the invention, there is provided a use of a matrix according to the invention as a tissue scaffold.

According to the invention, there is provided a matrix according to the invention for use as a tissue scaffold in repairing damaged tissue in a subject.

According to the invention, there is provided a cell culture or tissue culture comprising a matrix of the invention. The cell culture or tissue culture may comprise an incubator in which there is the matrix, cells and a nutrient medium.

Matrices of the invention may be applied to a medical device. Such matrices may be impregnated with cells, such as stem cells. For instance, matrices of the invention may be used to provide a layer or membrane on a medical device such as an implant or prostheses.

According to the invention, there is provided an implant or prosthesis comprising a matrix of the invention. The medical device (e.g. implant or prosthesis) may be manufactured from a plastics material or a metal.

Embodiments of the invention are now described, by way of example only, with reference to the accompanying drawings in which:

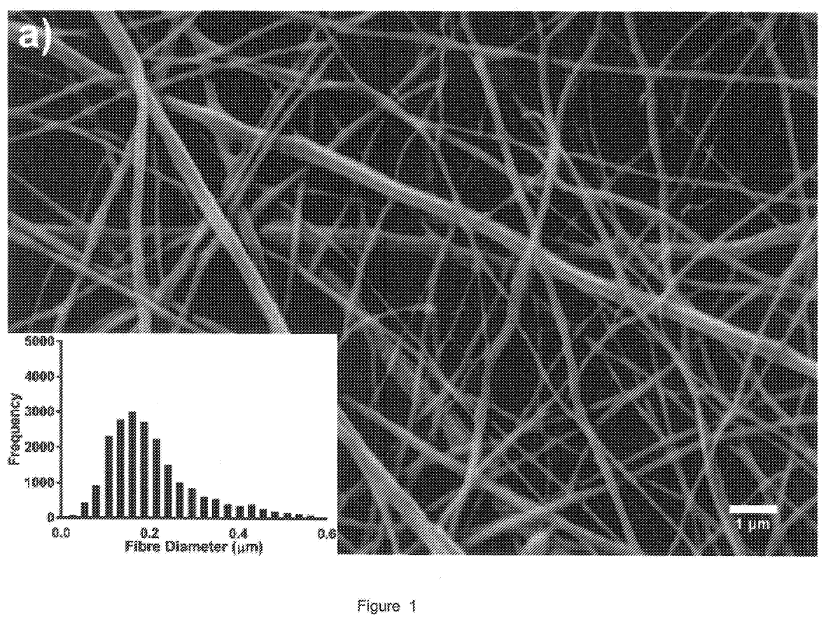

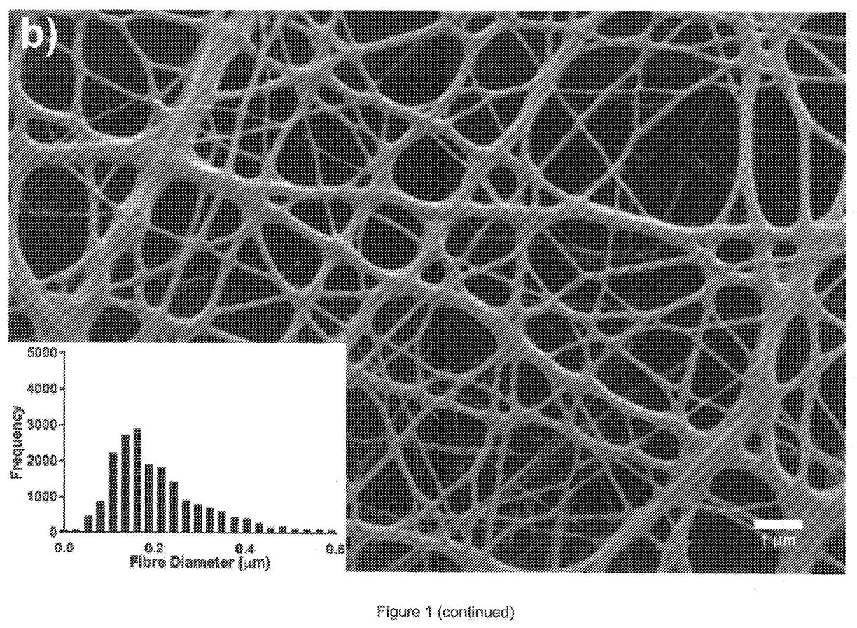

FIG. 1 shows SEM images of meshes a) 0% SurgihoneyRO™; b) 10% SurgihoneyRO™; c) 20% SurgihoneyRO™ and d) 30% SurgihoneyRO™, with corresponding insets of fibre distribution; e) Fibre diameter; and f) length;

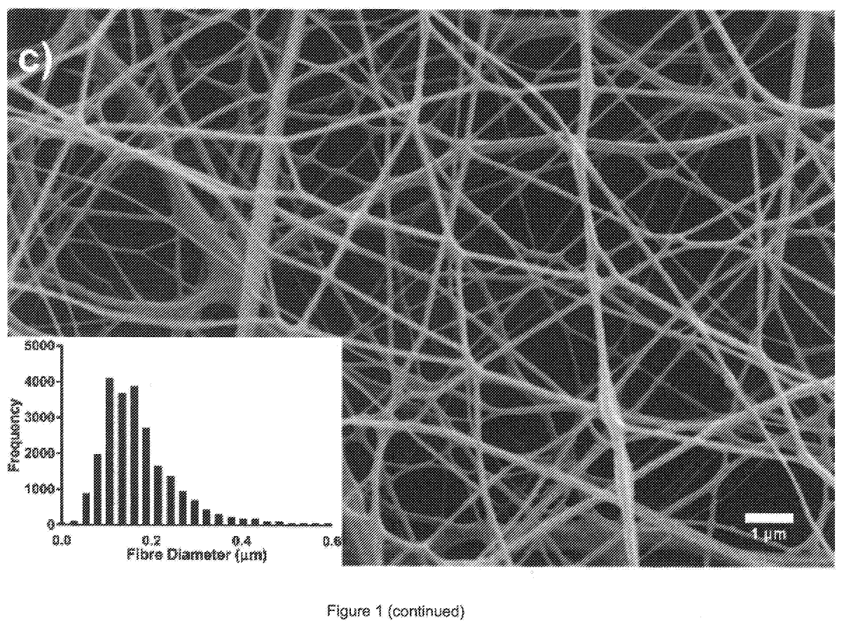

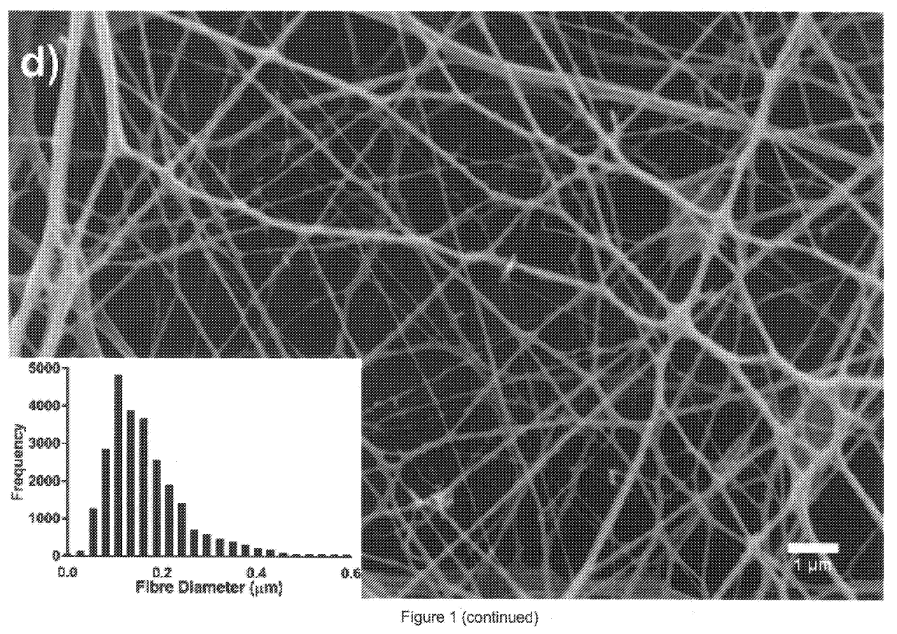

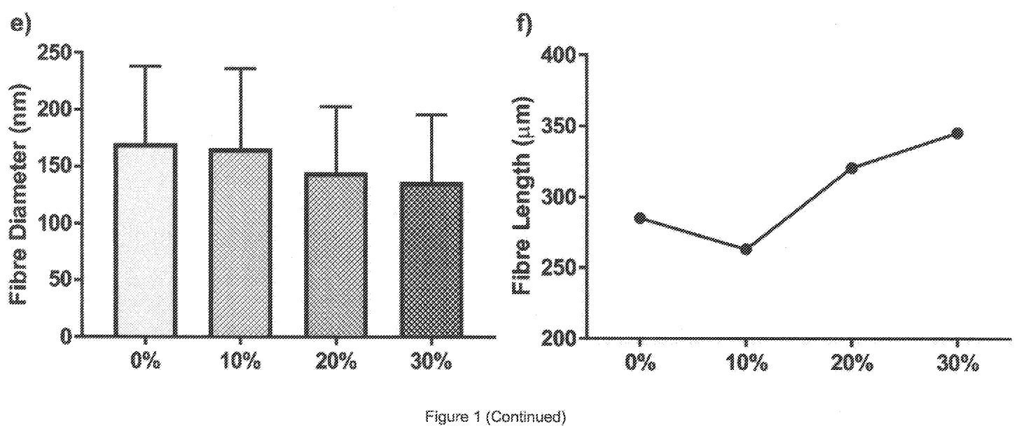

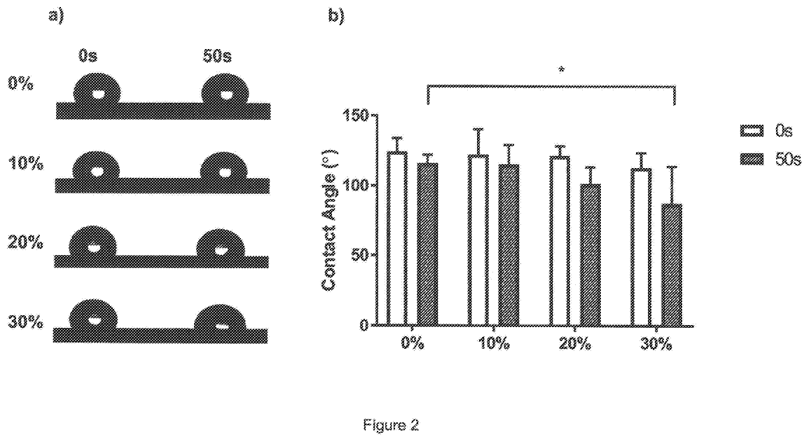

FIG. 2 shows water contact angle: a) Images of water droplet on mesh surfaces at 0 and 50 s. b) Contact angle measurements at 0 s and 50 s (*p<0.1);

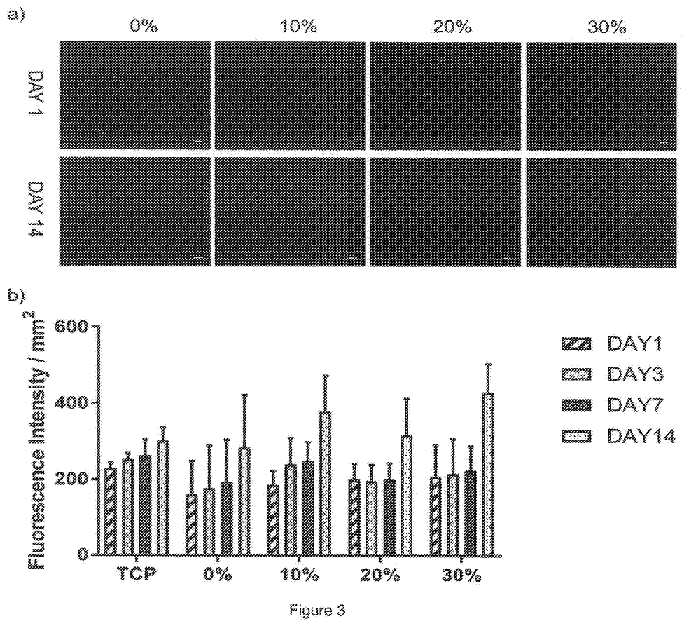

FIG. 3 shows a) Cell viability with Live/Dead staining at day 1 and 14 (scale bar 100 μm²); b) Cell proliferation with Alamar Blue assay at day 1, 3, 7, and 14 with NFI;

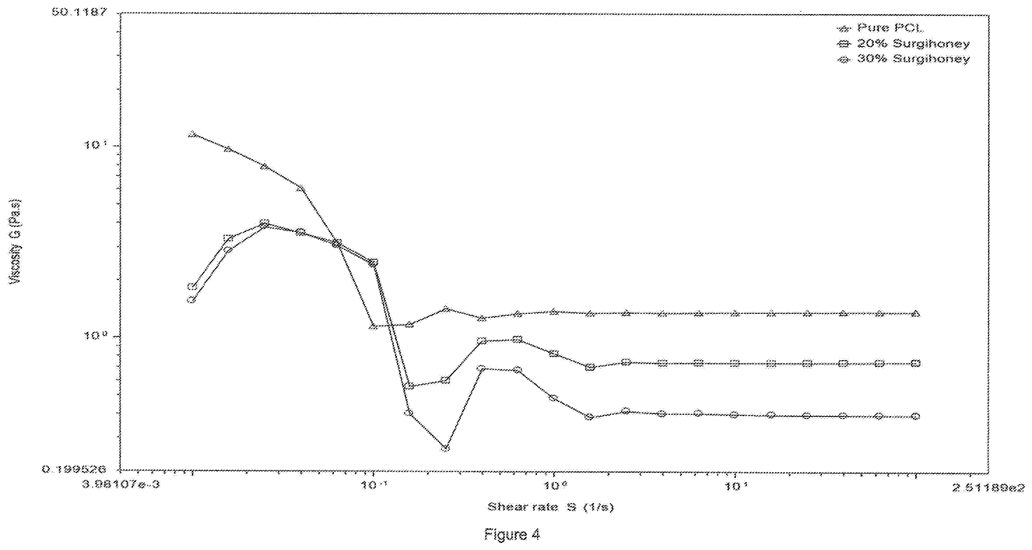

FIG. 4 shows a comparison of viscosities of solutions used to make meshes, as a function of shear rate (1/s);

FIG. 5 shows SEM images of electrospun meshes a) pure PCL, b) 20% SurgihoneyRO™, c) 30% SurgihoneyRO® and d) mean fiber diameter-honey concentration;

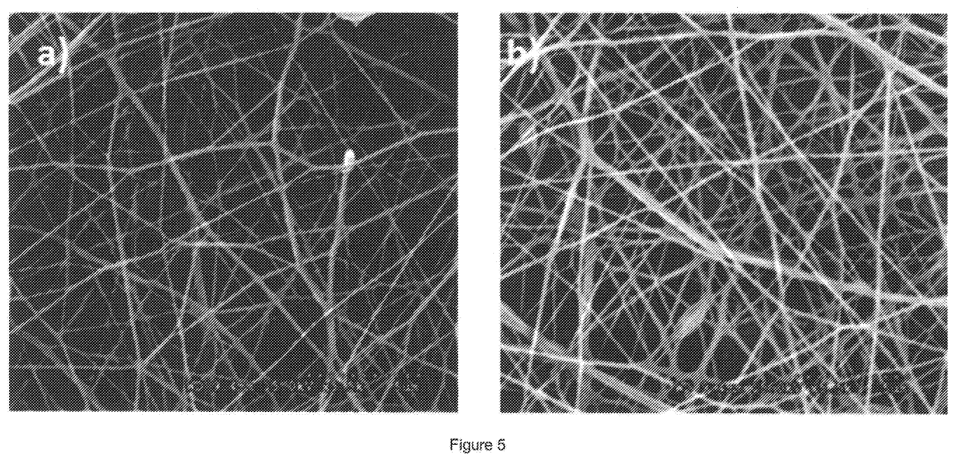

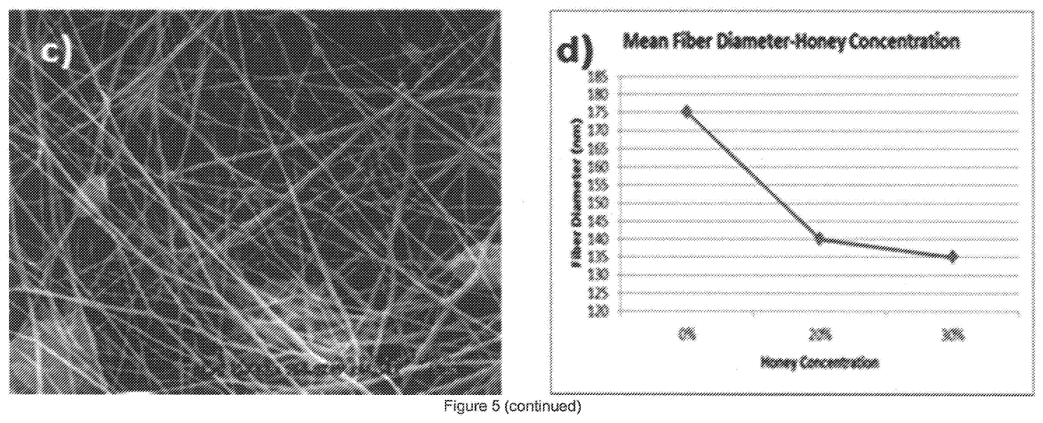

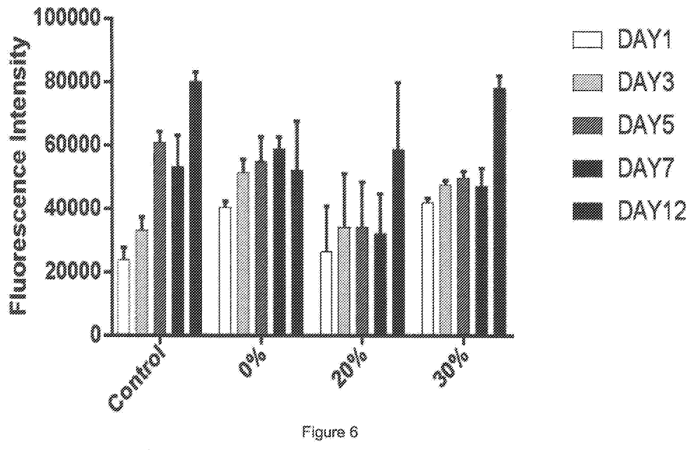

FIG. 6 shows a cell proliferation as a function of the fluorescence intensity on meshes;

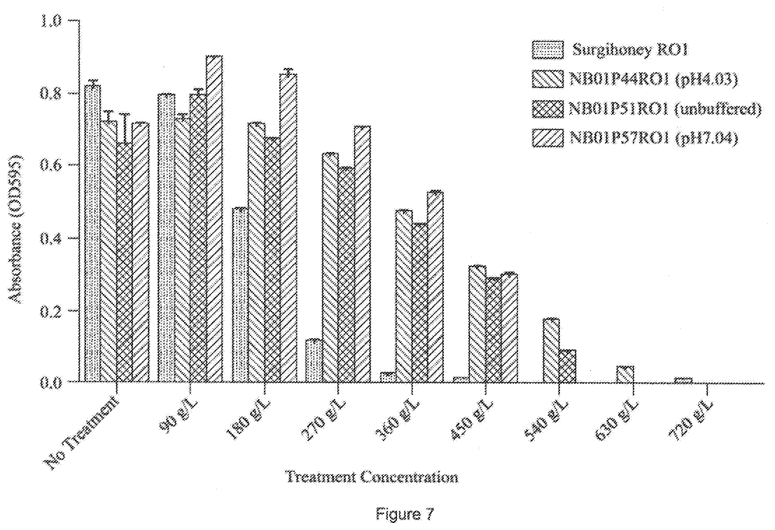

FIG. 7 is a graph showing the effect of compositions of the invention comprising glucose, glucose oxidase and fructose (SyntheticRO) on the growth of planktonic MRSA, compared to SurgihoneyRO, at various concentrations;

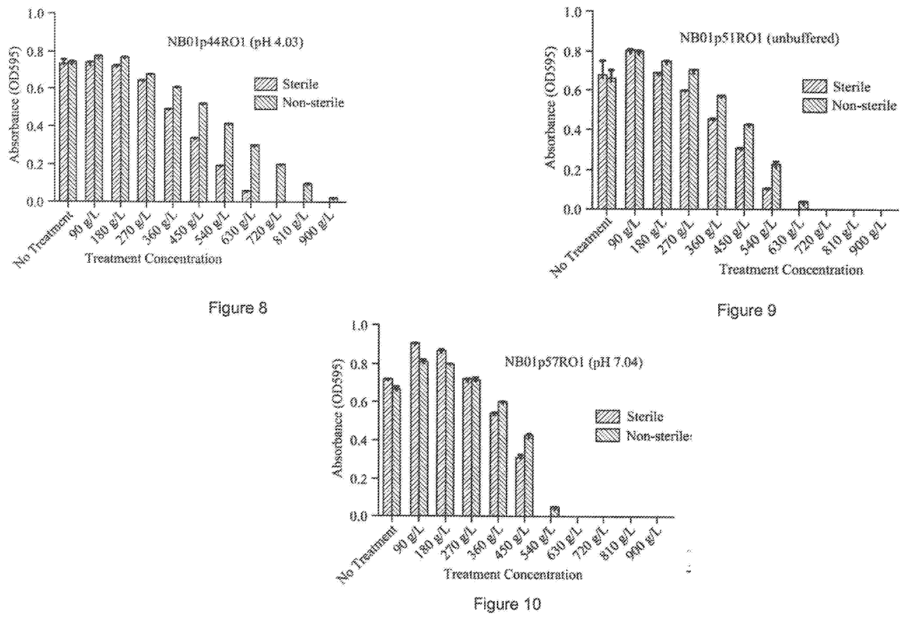

FIG. 8 is a graph showing the effect of sterile and non-sterile compositions of the invention comprising glucose, glucose oxidase and fructose (buffered at pH 4.03) on the growth of planktonic MRSA, at various concentrations;

FIG. 9 is a graph showing the effect of sterile and non-sterile compositions of the invention comprising glucose, glucose oxidase and fructose (unbuffered) on the growth of planktonic MRSA, at various concentrations;

FIG. 10 is a graph showing the effect of sterile and non-sterile compositions of the invention comprising glucose, glucose oxidase and fructose (buffered at pH 7.04) on the growth of planktonic MRSA, at various concentrations;

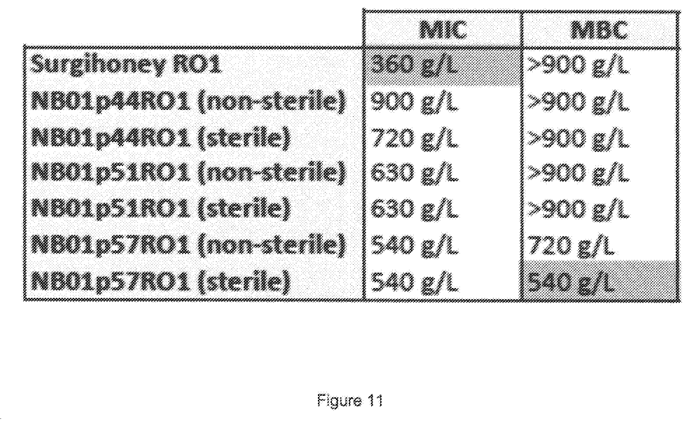

FIG. 11 is a table showing the effect of sterile and non-sterile compositions of the invention comprising glucose, glucose oxidase and fructose, on the MIC and MBC of planktonic MRSA, at various concentrations;

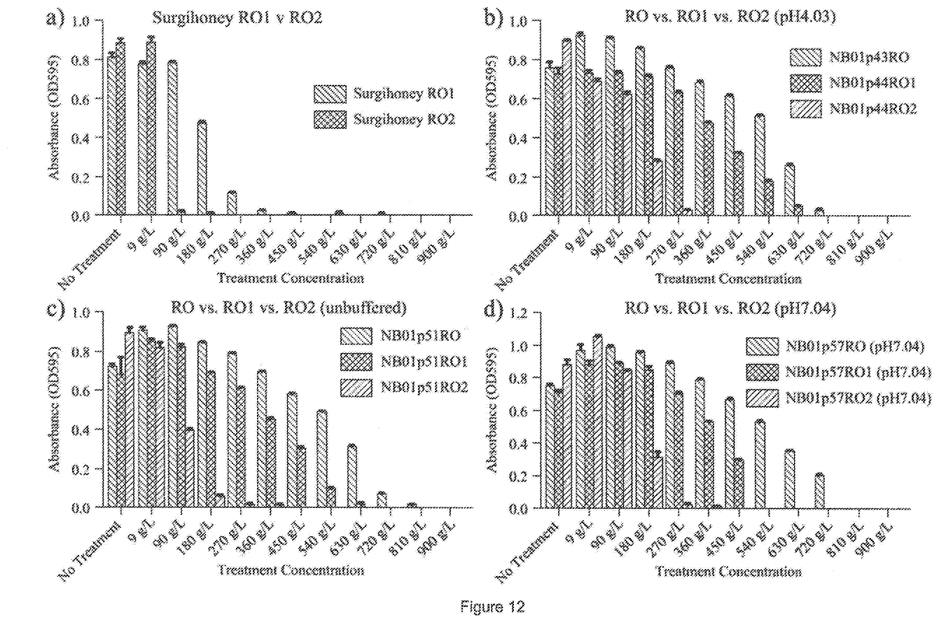

FIG. 12 shows the effect of compositions of the invention comprising glucose, glucose oxidase and fructose (SyntheticRO) on the growth of planktonic MRSA, compared to SurgihoneyRO™, at various concentrations;

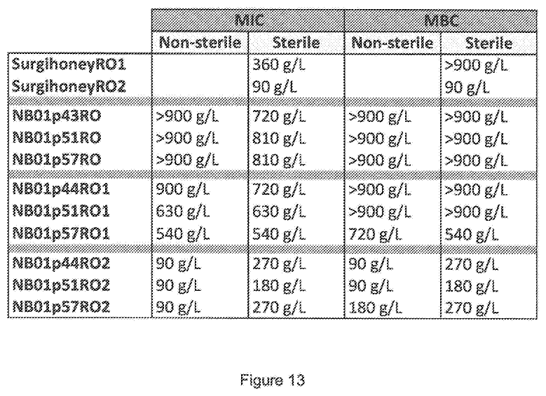

FIG. 13 shows the effect of SyntheticRO on the MIC and MBC of planktonic MRSA, compared to SurgihoneyRO™, at various concentrations;

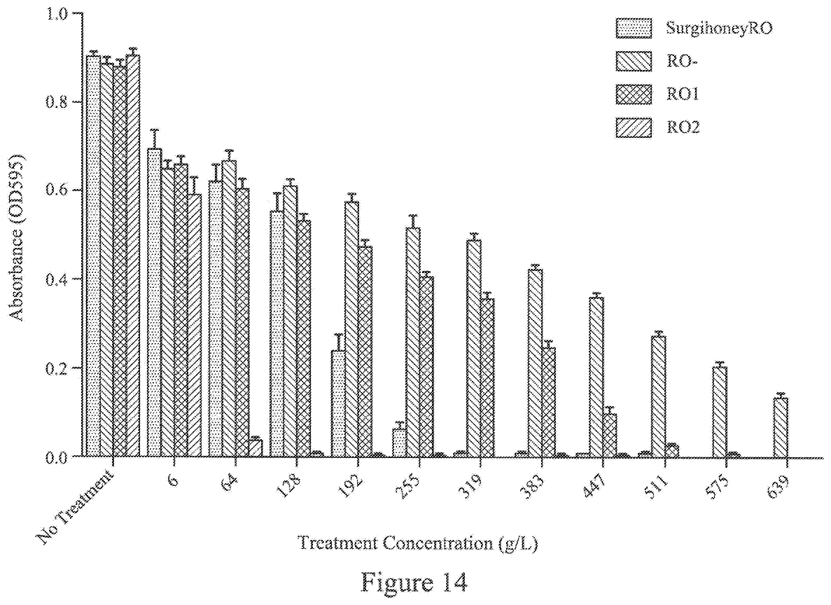

FIG. 14 shows the effect of SyntheticRO comprising glucose, glucose oxidase and fructose (SyntheticRO) on the growth of planktonic MSSA isolate;

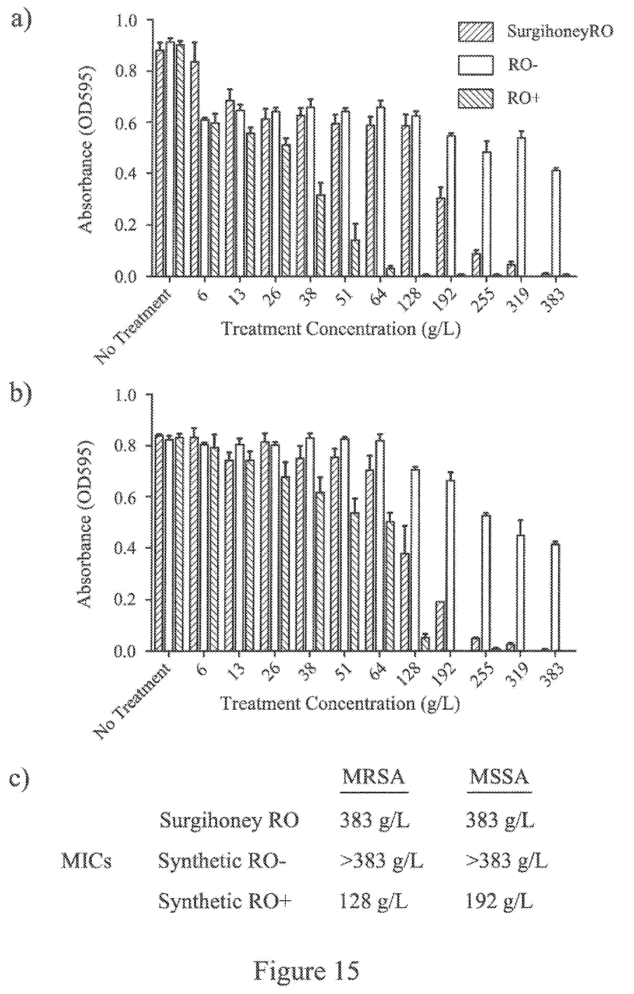

FIG. 15 (a and b) compares SyntheticRO with SurgihoneyRO™ using planktonic MRSA and MSSA, and FIG. 15 c is a table showing the MICs of a composition of the invention compared to SurgihoneyRO™. Planktonic MRSA and MSSA in vitro cultures were grown in the presence of the respective compositions for 18 hours, then the absorbance (OD₅₉₅) measured and compared to untreated cultures (n=6);

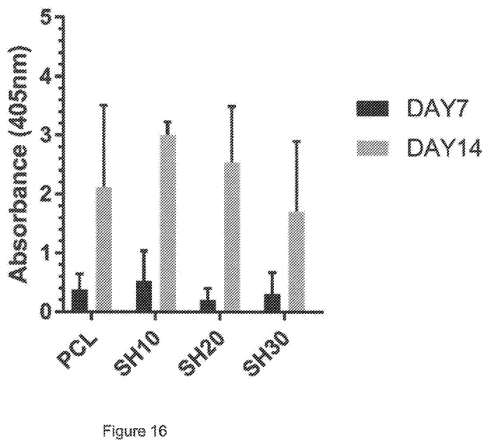

FIG. 16 shows the results of an Alkaline Phosphatase Activity (ALP) assay following addition of hADSCs to meshes containing SurgihoneyRO™

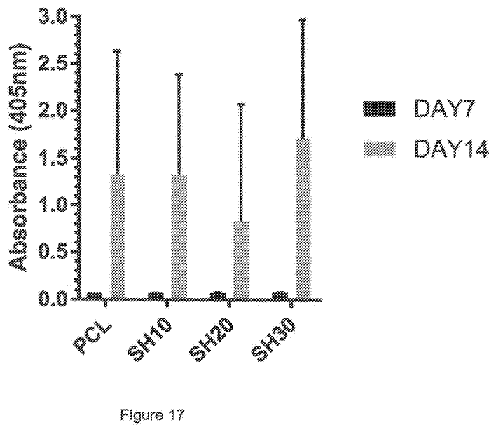

FIG. 17 shows the results of an Alazarin Red assay following addition of hADSCs to meshes containing SurgihoneyRO™;

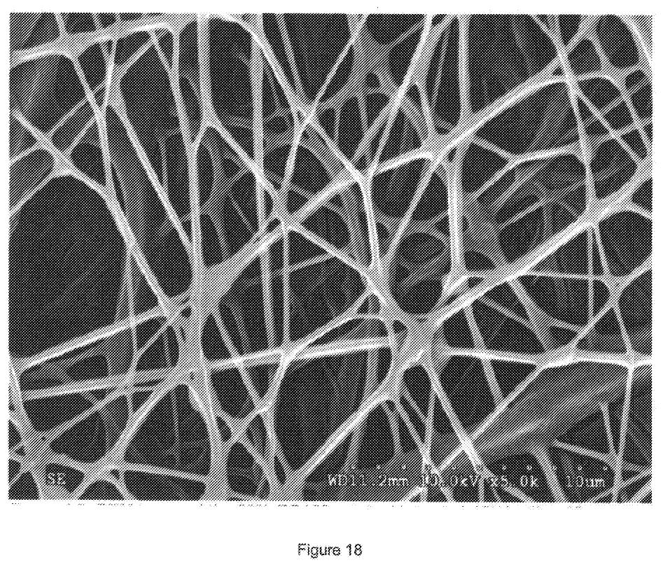

FIG. 18 shows a SEM image of a matrix containing RO100 with PCL (20% RO100);

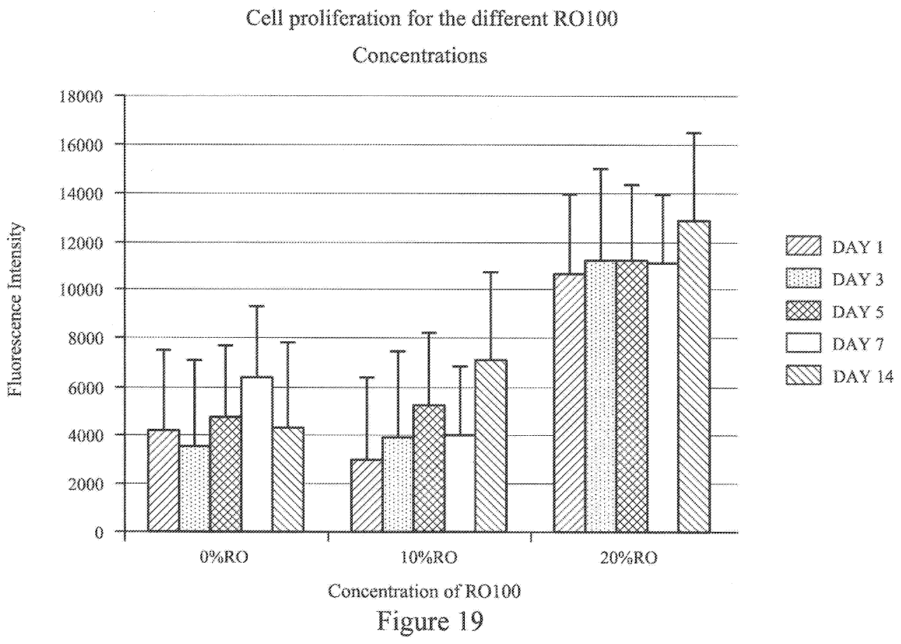

FIG. 19 shows the results of an Alamar Blue assay to assess cell viability on matrices containing RO100 and PCL, the matrices comprising different concentrations of RO100; and.

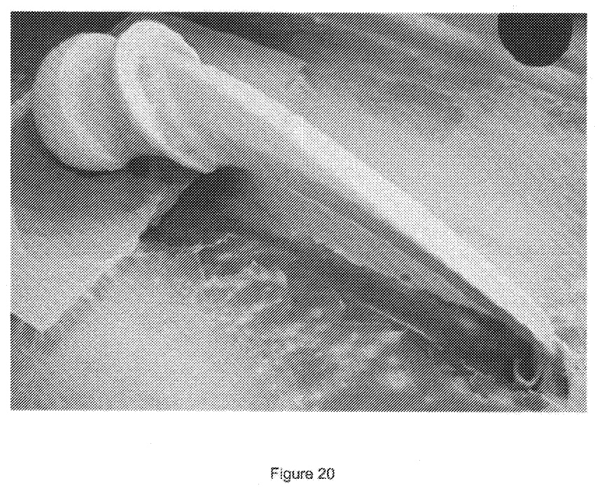

FIG. 20 shows a prosthetic hip joint coated with a PCL/RO100 mesh produced using electrospinning.

### EXAMPLE 1

### Materials and Methods

PCL (M_{w} 50,000 Da, CAPA 6500, Perstorp Caprolactones, UK) and SurgihoneyRO™ (Matoke Holdings, UK) meshes were produced using a solution electrospinning system (Profector, Spraybase, Ireland) consisting of high voltage power supply (from 0 kV to 30 kV), software to control a syringe pump system, stainless steel collector and stainless steel emitter*needle) with diameter 1 mm. Acetic acid (Fisher Scientific, UK) was used as a solvent to produce a range of PCL/SH concentrations whilst keeping the processing parameters constant (Table 1). All meshes were vacuumed dried for 24 hr to evaporate acetic acid.

TABLE 1
Material and processing conditions

| | | | | Total | | | Distance |
|---|---|---|---|---|---|---|---|
| | | | | concentration | | Flow | between |
| Honey | PC | | Acetic | of the | | Rate | needle and |
| Concentration | L | SurgihoneyRO ™ | Acid | mixture | Voltage | (mL/ | collector |
| (%) | (g) | (g) | (mL) | (%) | (kV) | min) | (mm) |
| 0 | 2 | 0 | 10 | 20 | 17 | 0.1 | 187 |
| 10 | 1.8 | 0.2 | 10 | 20 | 17 | 0.1 | 187 |
| 20 | 1.6 | 0.4 | 10 | 20 | 17 | 0.1 | 187 |
| 30 | 1.4 | 0.6 | 10 | 20 | 17 | 0.1 | 187 |

Meshes were characterised using scanning electron microscopy (SEM, Hitachi S-3000N, Japan) at an accelerating voltage of 15 kV. Samples were coated with platinum. The images were analysed using Fiji software with the DiameterJ plugin to assess fibre diameter and length [12].

The wettability of meshes (n=5) was determined through static contact angle measurement (KSV Cam 200, Finland). Images were obtained at 0 and 50 s after droplet formation and subsequently analysed using the Sessile drop technique.

In vitro biological characterisation of the meshes was performed using hADSCs (STEMPRO™, Thermo Fisher Scientific, USA). Cells were cultured with MesenPRO RS™ media containing 2% (v/v) growth supplement, 1% (v/v) glutamine, and 1% (v/v) penicillin/streptomycin until 80% confluence and harvested by the use of 0.05% trypsin-EDTA solution (Thermo Fisher Scientific, USA) at passage 7. Prior to cell seeding the meshes were sterilised using 80% ethanol for 2 hr and then dried overnight in a sterile laminar flow cabinet. 50,000 cells in 150 μL of media were seeded onto each mesh and incubated in a cell culture incubator (37° C., 5% CO₂, and 95% humidity) for 4 hr to allow cell attachment, before the addition of 450 μL fresh media. Cell proliferation was assessed at day 1, 3, 7, and 14 after cell seeding, using the resazurin assay (Alamar Blue) (Sigma-Aldrich, UK). On day 1 all samples (n=7) were transferred to a new 24-well plate to enable quantification of cell attachment and prevent unattached cells from influencing the result. Tissue culture plastic (TCP) was used as a control. At each time point, a 10% by volume (60 μL) of resazurin solution (0.01% (v/v)) was added to each sample and incubated for 4 hours. After incubation, 150 μL of each sample was transferred to a 96-well plate and the fluorescence intensity was measured (540 nm excitation/590 nm emission wavelength) with a plate reader (infinite 200, Tecan, Switzerland). Samples were washed twice in sterile PBS to remove the resazurin solution before the addition of fresh media. Cell culture media was changed every 3 days.

Cell viability was assessed using a Live/Dead Assay Kit (ThermoFisher Scientific, UK) at day 1 and day 14 according to the manufacturer's instructions. Cell culture media was removed from the samples (n=1) and TCP control and were washed with PBS twice before adding 500 μL of calcein-AM and EthD-1, 2 μm2 and 4 μm², respectively, PBS solution. The samples were then incubated for 25 min. Meshes were imaged with an inverted fluorescence microscope (Leica DMI6000 B, Leica Microsystems, Germany).

Results and Discussion

SEM images demonstrate the ability to successfully electrospin both PCL and POLISH meshes with nanoscale morphology mimicking the native ECM (FIG. 1). A distribution of fibre diameters ranging from 100-250 nm was observed (FIG. 1a-d inset).

Increasing the concentration of SH results in a decreasing fibre diameter (FIG. 1e). The average fibre diameter for 0, 10, 20, and 30% meshes are 170, 165, 144, and 136 nm, respectively. However, the fibre length increases with increasing SH concentration due to thinner fibre formation (FIG. 1f). The trend of increasing fibre length and decreasing diameter with increasing SH concentration is potentially related with a decrease in solution viscosity attributed to the reduced PCL content and higher SH concentration of the solution.

The wettability of the meshes as measured by water contact angle show that at 0 s after droplet formation all meshes present a hydrophobic surface with 0% having the highest contact angle, 124.31°, whilst 30% is the lowest (FIG. 2). The contact angle decreases with increasing concentration of SH at both 0 and 50 s. After 50 s meshes containing 30% of SH presents a contact angle of 86.8°. As a result, the addition of SH enhances the hydrophilicity of the electrospun meshes.

Cell seeding efficiency and the cell viability were calculated on day 1. The cell seeding efficiency was over 65% for all SH meshes when compared to the TCP control whilst attachment on PCL was lower. Cell viability, as measured by Live/Dead imaging, shows that approximately 95% of cells were alive (green) on the meshes on day 1 and by day 14 high cell viability was maintained with only few dead cells (red) observed, most likely due to the high cell density after two weeks of proliferation (FIG. 3a). These results indicate that cells are viable on the meshes containing SH.

All meshes supported cell proliferation as measured by Alamar Blue with a trend of increasing proliferation with higher SH concentration (FIG. 3b). After sterilisation all meshes contracted at different ratios, therefore, the diameter of all meshes was measured with a calliper and the fluorescence intensity was normalised to the area including for the TCP control. The normalised fluorescence intensity (NFI) of all meshes was comparable or greater than the TCP control by day 14. These results illustrate that the cells can attach and proliferate in the meshes. The trend of higher cell proliferation in SH meshes is potentially due to the more hydrophilic surface which allows improved cell spreading and serum proteins from the media attaching in the correct conformation. Furthermore, the SH may possibly be a source of nutrients for proliferating cells.

Conclusion

This study demonstrates the successful electrospinning of PCL meshes containing different concentrations of SurgihoneyRO™. The meshes exhibit nanoscale features resembling the ECM which show promising biological results with high cell viability and proliferation on all meshes. Subsequently, the meshes demonstrate suitability for tissue engineering applications,

### EXAMPLE 2

Electrospun meshes were produced in a similar manner as in Example 1 but with the following parameters.

| | | | | | Distance |
|---|---|---|---|---|---|
| Honey | | | | | between |
| Con- | | | | | needle |
| cen- | Weight | | | Flow | and |
| tration | of | Weight of | Voltage | Rate | collector |
| (%) | PCL (g) | Honey(g) | (kV) | (ml/min) | (mm) |
| 0 | 2 | 0 | 18 | 2 | 165 |
| 20 | 1.6 | 0.4 | 18 | 2 | 165 |
| 30 | 1.4 | 0.6 | 18 | 2 | 165 |

The viscosity of the prepared solutions was measured using the HR-2 Rheometer (TA Instruments, Elstree, UK). Viscosities were measured in triplicate for each concentration at room temperature with 0.5 mm zero gap. Results were obtained via the Trios Software.

Raw SurgihoneyRO™ samples were tested against the bacterium S. aureus using the minimum inhibitory concentration (MIC) method. MIC tests, also called basic microdilution method, determine the lowest concentration of a chemical compound that prevents the growth of a bacterium.

Scanning electron microscopy (SEM) was used to assess the morphology structure of the electrospun meshes. Meshes were coated with platinum sputtering during 40 seconds with the Cressington Sputter Coater 108 Auto (Watford, UK). High resolution images were taken using a HITACHI S-3000N (HITACHI, UK) electron microscope at an accelerating voltage of 15 kV. Obtained images were analysed using the DiameterJ software to determine the fibre diameter.

Electrospun meshes were also biologically assessed in terms of cell proliferation using human adipose-derived mesenchymal stem cells (hADMSC) (StemPro®, Thermo Fisher Scientific, UK). Five meshes were considered to determine the effect of each honey concentration on cell proliferation. Cell proliferation was evaluated using the AlamarBlue® assay kit (Sigma-Aldrich, UK) according to suppliers' protocol. AlamarBlue® reagent includes resazurin the active ingredient of AlamarBlue® reagent which is non-toxic and virtually non-fluorescent. When this reagent matched with living cells, it is reduced to resorufin which is a highly fluorescent molecule. Thus, cell proliferation can be quantitatively assessed. Cell proliferation was determined at day 1, 3, 5, 7 and 14. TCP was used as a control.

Antimicrobial Sensitivity Testing

Antibacterial properties of SurgihoneyRO™ were examined against S. aureus. The minimum inhibitory concentration of the SurgihoneyRO™ was about 6.25% which shows that it is able to inhibit the growth of the bacterium.

Rheological Behaviour of the PCL-SurgihoneyRO™ Solutions

FIG. 4 presents the variation of the viscosity of the PCL-SurgihoneyRO™ solutions with acetic acid as a function of shear rate.

Results show that the viscosity decreases by increasing the concentration of SurgihoneyRO™ and decreasing PCL concentration in the solutions. For low shear rates (0.39 s⁻¹), the viscosity decreases by increasing the shear rate (shear thinning behaviour). For high shear rates the viscosity remains constant with the increase of the shear rate (Newtonian behaviour). In this last regime, the viscosity of the PCL solution is approximately 1.34 Pa·s while the viscosity of PCL/SurgihoneyRO™ (30%) is approximately 0.42 Pa·s. The initial viscosity of PCL solution is around 10.74 Pa·s and PCL/SurgihoneyRO™ (30%) is approximately 1.55 Pa·s.

Morphological Analysis of the Meshes

SEM images of nonwoven meshes were taken with 7500 magnification at 5 μm² scale (FIG. 5). Nonwoven PCL and PCL-SurgihoneyRO™ meshes exhibit a distribution of fibre diameters ranging from 100 nm to 250 nm. Meshes produced with different concentrations of SurgihoneyRO™ show similar fibre diameter distribution trend. However, the meshes have different fibre diameter values according to the concentration of the SurgihoneyRO™. The fibre diameter decreases with increasing the amount of SurgihoneyRO™ in meshes as follows: 175 nm, 141 nm, and 136 nm for meshes containing 0%, 20% and 30% SurgihoneyRO™, respectively (FIG. 5d).

Biological Results

Cell proliferation tests were performed over 14 days. Following an initial seeding density of 50,000 cells per well, all meshes presented an increase in fluorescence intensity at each time point up to 14 days in culture media (FIG. 6). The fluorescence intensity of cells in the treated well plate as positive controls showed an increase as well. Meshes without SurgihoneyRO™ exhibit high cell proliferation up to day 7. However, at day 14 the meshes containing 30% of SurgihoneyRO™ present the highest fluorescence intensity. Additionally, pure PCL (not containing SurgihoneyRO™) meshes have lower fluorescence intensity than the meshes containing SurgihoneyRO™. These results show that the fabrication of electrospun meshes with SurgihoneyRO™ enhances the cell proliferation. Moreover, it also shows that acetic acid does not affect cell proliferation negatively. This means that SurgihoneyRO™ provides a suitable environment for cell proliferation and adding SurgihoneyRO™ into meshes does not have a negative effect for cell proliferation.

Conclusions

The raw SurgihoneyRO™ has a good antibacterial property against the bacteria of S. aureus which are commonly found on a skin wound, at the concentration of 6.25%. PCL and SurgihoneyRO™ mixture are able to be spun together. Therefore, these antibacterial properties show promising application in tissue engineering applications and utilisation in electrospun meshes. Composite PCL-SurgihoneyRO™ meshes have been fabricated using solution electrospinning process. Meshes have smaller fibre diameter size range from 100 to 250 nm, mimicking the scale of the extracellular matrix (ECM), which means they are suitable for wound dressings. On the other hand, increasing the SurgihoneyRO™ concentration in the meshes leads to decreasing of fibre diameter, and also the same effect observed for the viscosity. Biological tests using hADSC show that all produced meshes allow cell attachment and proliferation. Moreover, results show at day 14, better results were obtained for meshes containing 30% SurgihoneyRO™.

### EXAMPLE 3—SYNTHETIC HONEY COMPOSITIONS (SYNTHETICRO)

Samples with batch number “RO” contain no glucose oxidase.

Samples with batch number “RO1” contain 50 ppm glucose oxidase.

Samples with batch number “RO2” contain 1000 ppm glucose oxidase.

A. pH 4.03 buffered samples

A1. Batch no NB01p43RO
Non sterile

| Material | Weight fraction |
|---|---|
| Fructose | 52.0% |
| Glucose | 31.0% |
| 50 mMol Citric acid/NaOH | 17.0% |
| buffer pH 4.03 | |

Description

Non sterile base buffered saccharide solution.

A2. Batch no NB01p43RO
Sterile

| Material | Weight fraction |
|---|---|
| Fructose | 52.0% |
| Glucose | 31.0% |
| 50 mMol Citric acid/NaOH | 17.0% |
| buffer pH 4.03 | |

Description Sterile base buffered saccharide solution

A3. Batch no NB01p44RO1
Non sterile

| Material | Weight fraction |
|---|---|
| Fructose | 52.0% |
| Glucose | 31.0% |
| 50 mMol Citric acid/NaOH | 17.0% |
| buffer pH 4.03 | |

Description

Non sterile base buffered RO1 saccharide solution.

A4, Batch no NB01p44RO1
Sterile

| Material | Weight fraction |
|---|---|
| Fructose | 52.0% |
| Glucose | 31.0% |
| 50 mMol Citric acid/NaOH buffer pH 4.03 | 17.0% |

Description

Sterile base buffered RO1 saccharide solution

A5, Batch no NB01p44RO2
Non sterile

| Material | Weight fraction |
|---|---|
| Fructose | 52.0% |
| Glucose | 31.0% |
| 50 mMol Citric acid/NaOH buffer pH 4.03 | 17.0% |

Description

Non sterile base buffered RO2 saccharide solution,

A6, Batch no NB01p43RO2
Sterile

| Material | Weight fraction |
|---|---|
| Fructose | 52.0% |
| Glucose | 31.0% |
| 50 mMol Citric acid/NaOH buffer pH 4.03 | 17.0% |
| GOX enzyme | N/A |

Description Sterile base buffered RO2 saccharide solution

B. Unbuffered Samples

B1. Batch no NB01p51RO
Non sterile

| Material | Weight fraction |
|---|---|
| Fructose | 52.0% |
| Glucose | 31.0% |
| Water | 17.0% |

Description

Non sterile base buffered saccharide solution.

B2. Batch no NB01p51RO
Sterile

| Material | Weight fraction |
|---|---|
| Fructose | 52.0% |
| Glucose | 31.0% |
| Water | 17.0% |

Description Sterile base buffered saccharide solution

B3. Batch no NB01p51RO1
Non sterile

| Material | Weight fraction |
|---|---|
| Fructose | 52.0% |
| Glucose | 31.0% |
| Water | 17.0% |

Description

Non sterile base buffered RO1 saccharide solution.

B4. Batch no NB01p51RO1
Sterile

| Material | Weight fraction |
|---|---|
| Fructose | 52.0% |
| Glucose | 31.0% |
| Water | 17.0% |

Description

Sterile base buffered RO1 saccharide solution

B5. Batch no NB01p51RO2
Non sterile

| Material | Weight fraction |
|---|---|
| Fructose | 52.0% |
| Glucose | 31.0% |
| Water | 17.0% |

Description

Non sterile base buffered RO2 saccharide solution

B6. Batch no NB01p51RO2
Sterile

| Material | Weight fraction |
|---|---|
| Fructose | 52.0% |
| Glucose | 31.0% |
| Water | 17.0% |

Description
Sterile base buffered RO2 saccharide solution

C. pH 7.04 buffered samples

C1. Batch no NB01p57RO
Non sterile

| Material | Weight fraction |
|---|---|
| Fructose | 52.0% |
| Glucose | 31.0% |
| 50 mMol Citric acid/NaOH buffer pH 7.04 | 17.0% |

Description

Non sterile base buffered saccharide solution.

C2. Batch no NB01p57RO
Sterile

| Material | Weight fraction |
|---|---|
| Fructose | 52.0% |
| Glucose | 31.0% |
| 50 mMol Citric acid/NaOH buffer pH 7.04 | 17.0% |

Description

Sterile base buffered saccharide solution

C3. Batch no NB01p57RO1
Non sterile

| Material | Weight fraction |
|---|---|
| Fructose | 52.0% |
| Glucose | 31.0% |
| 50 mMol Citric acid/NaOH buffer pH 7.04 | 17.0% |

Description

Non sterile base buffered RO1 saccharide solution.

C4. Batch no NB01p57RO1
Sterile

| Material | Weight fraction |
|---|---|
| Fructose | 52.0% |
| Glucose | 31.0% |
| 50 mMol Citric acid/NaOH buffer pH 7.04 | 17.0% |

Description

Sterile base buffered RO1 saccharide solution

C5. Batch no NB01p57RO2
Non sterile

| Material | Weight fraction |
|---|---|
| Fructose | 52.0% |
| Glucose | 31.0% |
| 50 mMol Citric acid/NaOH buffer pH 7.04 | 17.0% |

Description

Non sterile base buffered RO2 saccharide solution.

C6. Batch no NB01p57RO2

| Material | Weight fraction |
|---|---|
| Fructose | 52.0% |
| Glucose | 31.0% |
| 50 mMol Citric acid/NaOH buffer pH 7.04 | 17.0% |

Description

Sterile base buffered RO2 saccharide solution

### EXAMPLE 4—EFFICACY OF SYNTHETIC HONEY COMPOSITIONS AGAINST PLANKTONIC MRSA

MIC and MBC were assessed for the RO1 samples (containing 50 ppm glucose oxidase) and compared to SurgihoneyRO™ (also containing 50 ppm glucose oxidase). See Andrews J. M. Journal of Antimicrobial Chemotherapy (2001) 48, suppl. S1, 5-16.

The results are shown in FIGS. 7 to 11.

The results show that, like SurgihoneyRO™, synthetic compositions containing glucose, glucose oxidase and fructose are able to inhibit microbial growth.

Out of all of synthetic compositions, the synthetic composition buffered at pH7.04 had the most effective MIC. Sterilised compositions were more effective than non-sterilised compositions, and synthetic composition buffered at pH7.04 synthetic had the most effective MBC when compared to other synthetic compositions and even when compared to SurgihoneyRO™.

FIGS. 12 (a to d) and 13 show MIC and MBC results including SurgihoneyRO2 samples and synthetic RO2 samples.

pH 7.04 formulations were tested against a planktonic MSSA isolate. FIG. 14 shows the results obtained.

The synthetic RO2 composition was selected for further investigation. FIG. 15 (a, b and c) show SyntheticRO (RO2; pH7.04) compared to SurgihoneyRO™ using planktonic phenotype. RO— indicates a product lacking enzyme activity.

### EXAMPLE 5—CELL EXPERIMENTS

In vitro biological characterisation of the meshes was performed using hADSCs (STEMPRO™, Thermo Fisher Scientific, USA). Cells were cultured with MesenPRO RS™ media containing 2% (v/v) growth supplement, 1% (v/v) glutamine, and 1% (v/v) penicillin/streptomycin until 80% confluence and harvested by the use of 0.05% trypsin-EDTA solution (Thermo Fisher Scientific, USA) at passage 7. Prior to cell seeding the meshes were sterilised using 80% ethanol for 2 hr and then dried overnight in a sterile laminar flow cabinet. 15,000 cells in 150 μL of media were seeded onto each mesh and incubated in a cell culture incubator (37° C., 5% CO₂, and 95% humidity) for 4 hr to allow cell attachment, before the addition of 350 μL fresh media.

Meshes containing SurgihoneyRO™ and PCL (Surgihoney: 10%, 20% and 30%) were assessed using the following cell assays.

Alkaline Phosphatase Activity (ALP)

To investigate the osteogenic differentiation of hADSCs seeded on the meshes, alkaline phosphatase enzyme activity was observed using a colorimetric assay (SensoLYTE® Pnpp Alkaline Phosphatase Assay Kit, AnaSpec, Fremont, Calif., USA), using manufacturer's protocol.

Firstly, solutions were prepared which were used in the experiment.
1×ALP assay buffer solution was prepared from 10×ALP assay buffer solution (Manufacturer provided).
1 ml of 10×ALP assay buffer solution mix with 9 ml of deionized water to make 1×ALP assay buffer.
0.2% v/v Triton X-100 was prepared. 20 μl was added to 10 ml of 1×ALP assay buffer to make 0.2% v/v Triton X-100
1. Samples (n=3) were transferred to 24 well plates
2. Samples were washed twice with ALP dilutions assay buffer.
3, Samples were transferred from 24-well plates to 1.5 ml Eppendorf tubes.
4. 250 μl 1×ALP assay buffer containing 0.2% v/v Triton X-100 was added to each tube.
5. Each sample was vortexed (in the Eppendorf tubes) for 1 min.
6. All samples (in the Eppendorf tubes) were centrifuged at 1700×g for 15 mins at 4° C.
7. 50 μl supernatants were taken from the each tube and transferred to 96-well plates.
8. 50 μl pNPP (manufacturer provided) was added into supernatants in the 96-well plates and left at room temperature in the dark by covering with aluminium foil for 1 h.
9. 50 μl stop solution (manufacturer provided) was added to into 96 well plate, to form a 150 μl solution each well.
10. Absorbance was measured at 405 nm.

FIG. 16 shows the results at day 7 and day 14. It is noted that alkaline phosphatase activity is an early marker of bone development.

Alizarin Red

To investigate the mineralisation of hADSCs seeded on the meshes, Alizarin red-S(ARS) (Sigma Aldrich, Dorset, UK) assay was used.
1. 0.2% w/v ARS solution was prepared using ARS powder and distilled water. This solution was covered with foil to protect it from the light.
2. Meshes were transferred to 24 well plate and then washed with PBS twice.
3. Meshes were immersed in 10% Formaldehyde solution 15 mins at room temperature.
4. Formaldehyde solution was removed and samples washed with deionized water three times.
5. 0.2% ARS staining solution was added to meshes until covering the meshes.
6. 24-well plates covered were covered with foil and left for 40 mins at the room temperature in the dark.
7. Samples were washed with deionized water 5 times (each times after a 5 min wait).
8. Meshes were transferred to 1.5 ml Eppendorf tubes and 800 μl 10% Acetic Acid solution was added into these tubes, and the tubes were shaken gently at room temperature for 30 mins.
9. The solutions were transferred to new Eppendorf tubes.
10. The solutions in new tubes were vortexed for 30 seconds each.
11. The solutions were heated at 85° C. for 10 mins. To avoid evaporation, the tubes were sealed with film.
12. The tubes were kept in the freezer for 5 mins to cool down.
13. The tubes were centrifuged at 1700×g for 15 mins.
14. After centrifugation, 150 μl supernatants transferred to 96-well plates and absorbance was measured at 405 nm.

FIG. 17 shows results at day 7 and day 14. Alizarin Red stains calcium which is formed during osteogenic differentiation and bone development. All samples show calcium formation.

### EXAMPLE 7—MESH PRODUCTION FOR NON-HONEY-BASED, SYNTHETIC COMPOSITION

This example utilised a synthetic composition (referred to as RO100), comprising glucose (31%, by weight), fructose (52%, by weight). Water (17%, by weight) and glucose oxidase (0.5% by weight).

RO100-based matrices were formulated by electrospinning compositions containing PCL, R0100 and acetic acid. The relative amounts of RO100 and PCL were varied (10%, 20% and 30% RO100).

It was found that RO100-based matrices could be formed when using, for example, a distance between needle and collector of 15 cm, a flow rate of 0.1 ml/minute, a voltage of 15 kV or 10 kV and a processing time of 20 minutes.

FIG. 18 shows an SEM image of a 20% RO100 mesh obtained using a voltage of 10 kV.

The mean fiber diameter for all produced samples was within 180-300 nm, with porosity (measured using DiameterJ software) between 0.46 and 0.59.

### EXAMPLE 8—CELL VIABILITY TEST

Proliferation of hADSCs was detected on RO100-based matrices using the AlamarBlue assay (See Examples 1 and 2). The results are shown in FIG. 19. It is noted that over the 14 day period, the increase in stem cell count was higher for both the 10% RO100 matrix (138%) and the 20% RO100 matrix (18%) compared to a matrix comprising PCL but without RO100 (3%).

### EXAMPLE 9—COATING MEDICAL DEVICES

FIG. 20 shows a prosthetic hip joint coated with a PCL/RO100 mesh produced by electrospinning a solution comprising PCL, acetic acid and RO100.

### REFERENCES

J. R. Dias, P. L. Granja, P. J. Bártolo, Advances in electrospun skin substitutes, Progress in Materials Science 84 (2016) 314-334.
R. F. Pereira, P. J. Bártolo, Traditional Therapies for Skin Wound Healing, Advances in Wound Care 5(5) (2016) 208-229.
F. Gottrup, J. Apelqvist, P. Price, Outcomes in controlled and comparative studies on non-healing wounds: recommendations to improve the quality of evidence in wound management, J Wound Care 19(6) (2010) 237-68.
K. Alexiadou, J. Doupis, Management of Diabetic Foot Ulcers, Diabetes Therapy 3(1) (2012) 4,
P. Zahedi, I. Rezaeian, S.-O. Ranaei-Siadat, S.-H. Jafari, P. Supaphol, A review on wound dressings with an emphasis on electrospun nanofibrous polymeric bandages, Polymers for Advanced Technologies 21(2) (2010) 77-95.
Z. H. Israili, Antimicrobial properties of honey, American journal of therapeutics 21(4) (2014) 304-23.
M. D. Mandal, S. Mandal, Honey: its medicinal property and antibacterial activity, Asian Pacific Journal of Tropical Biomedicine 1(2) (2011) 154-160.
M. Dryden, G. Lockyer, K. Saeed, J. Cooke, Engineered honey: In vitro antimicrobial activity of a novel topical wound care treatment, Journal of Global Antimicrobial Resistance 2(3) (2014) 168-172.
J. Cooke, M. Dryden, T. Patton, J. Brennan, J. Barrett, The antimicrobial activity of prototype modified honeys that generate reactive oxygen species (ROS) hydrogen peroxide, BMC Research Notes 8(1) (2015) 20.
N. R. Love, Y. Chen, S. Ishibashi, P. Kritsiligkou, R. Lea, Y. Koh, J. L. Gallop, K. Dorey, E. Amaya, Amputation-induced reactive oxygen species are required for successful Xenopus tadpole tail regeneration, Nature Cell Biology 15 (2013) 222.
C. Dunhill, T. Patton, J. Brennan, J. Barrett, M. Dryden, J. Cooke, D. Leaper, N. T. Georgopoulos, Reactive oxygen species (ROS) and wound healing: the functional role of ROS and emerging ROS-modulating technologies for augmentation of the healing process, International Wound Journal 14(1) (2017) 89-96.
N. A. Hotaling, K. Bharti, H. Kriel, C. G. Simon, DiameterJ: A validated open source nanofiber diameter measurement tool, Biomaterials 61 (2015) 327-338.
J. Wu, Y. Hong: Enhancing cell infiltration of electrospun fibrous scaffolds in tissue regeneration, Bioactive Materials 1 (2016) 56-64
J Rnjak-Kovacina & A. S. Weiss: Increasing the Pore Size of Electrospun Scaffolds, Tissue Engineering: Part B 17(5) (2011) 365-372
M Haeri & M Haeri: lmageJ Plugin for Analysis of Porous Scaffolds used in Tissue Engineering, Journal of Open Research Software, 3: el

1. A matrix for use as a tissue scaffold, comprising a purified enzyme that is able to convert a substrate to release hydrogen peroxide and a substance that includes a substrate for the enzyme.

2. A matrix according to claim 1, comprising one or more fibers.

3. A matrix according to claim 2, comprising one or more nanofibers.

4. A matrix according to any preceding claim, comprising a polymer.

5. A matrix according to claim 4, wherein the polymer is polycaprolactone.

6. A matrix according to any preceding claim, formed by electrospinning.

7. A matrix according to any preceding claim, wherein the enzyme is at least 95% pure or is pharmaceutical grade.

8. A matrix according to any preceding claim, wherein the matrix does not comprise sufficient free water to allow the enzyme to convert the substrate.

9. A matrix according to any preceding claim, wherein the enzyme is an oxidoreductase enzyme, preferably wherein the enzyme is glucose oxidase.

10. A matrix according to any preceding claim, wherein the substance comprises a purified substrate for the enzyme.

11. A matrix according to claim 10, wherein the substrate is at least 95% pure or is pharmaceutical grade.

12. A matrix according to any of claims 10 to 11, wherein the substance comprises a solute with a solubility greater than or equal to 100 g/100 g water at 20° C. and 1 atm, preferably greater than or equal to 200 g/100 g water at 20° C. and 1 atm, more preferably greater than or equal to 300 g/100 g water at 20° C. and 1 atm.

13. A matrix according to claim 12, wherein the solute is a sugar or sugar derivative, preferably wherein the solute is fructose,

14. A matrix according to claim 12 or claim 13, wherein the solute is a purified solute, preferably at least 95% pure or pharmaceutical grade.

15. A matrix according to any of claims 1 to 9, wherein the substance is, or comprises, an unrefined natural substance.

16. A matrix according to any preceding claim, wherein the substance is or comprises honey.

17. A matrix according to any preceding claim which is sterile.

18. A matrix according to any preceding claim which is bioabsorbable or biodegradable.

19. A matrix according to claim 2 or any claim dependent on claim 2 which has a fiber diameter range within 10 to 500 nm, 50 to 300 nm or 100 to 250 nm.

20. A matrix according to claim 2 or any claim dependent on claim 2, with a mean fiber diameter of 10 to 500 nm, 50 to 300 nm, 100 to 250 nm, or 100 to 200 nm.

21. A matrix according to claim 2 or any claim dependent on claim 2, with a mean pore size of 2 μm to 1000 μm, 2 μm to 500 μm, 2 μm to 250 μm or 2 μm to 50 μm.

22. A matrix according to claim 2, or any claim dependent on claim 2, with a mean pore size of 5 μm to 1000 μm, 5 μm to 500 μm, 5 μm to 250 μm, 5 μm to 100 μm, or 5 μm to 50 μM.

23. A matrix according to claim 2 or any claim dependent on claim 2 with a mean pore size of 10 μm to 1000 μm, 10 μm to 500 μm, 10 μm to 250 μm, 10 μm to 100 μm, or 10 to 50 μm.

24. A matrix according to claim 2 or any claim dependent on claim 2, with a mean pore size of 50 μm to 1000 μm, 50 μm to 500 μm or 50 μm to 250 μm.

25. A matrix according to claim 2, or any claim dependent on claim 2, with a mean pore size of 100 μm to 1000 μm, 100 μm to 500 μm, or 100 μm to 250 μm.

26. A matrix according to claim 2 or any claim dependent on claim 2, with a mean pore size of 200 μm to 1000 μm or 200 μm to 500 μm.

27. A matrix according to any preceding claim with a porosity of at least 40%, at least 50%, at least 80%, at least 85%, at least 90% or at least 95%.

28. A matrix according to any preceding claim, which comprises substantially no hydrogen peroxide, or no detectable hydrogen peroxide.

29. A matrix according to any preceding claim, which comprises no added peroxidase, substantially no peroxidase, or is essentially free of peroxidase.

30. A matrix according to any preceding claim, which comprises no added zinc oxide, substantially no zinc oxide, or is essentially free of zinc oxide.

31. A matrix according to any preceding claim, in combination with at least one tissue cell.

32. A matrix according to claim 31, impregnated or seeded with the at least one cell,

33. A matrix according to claim 31 or claim 32, wherein the at least one cell comprises a skin cell.

34. A matrix according to any of claims 31 to 33, wherein the at least one cell is a stem cell.

35. A matrix according to any of claims 31 to 34, wherein the at least one cell is a human adipose-derived stem cell (hADSC).

36. A matrix according to any preceding claim with a water activity (a_{w}) of 0.8 or less,

37. A matrix according to claim 36, with a water activity of 0.2 to 0.8, preferably 0.3 to 0.7.

38. A matrix according to any preceding claim that does not comprise an unrefined natural substance.

39. A matrix according to any preceding claim, which does not comprise honey.

40. A matrix according to any preceding claim, which is pharmaceutical grade, or wherein its components are pharmaceutical grade.

41. A method of repairing damaged tissue in a subject comprising administering a matrix as defined in any of claims 1 to 40, to the damaged tissue.

42. A method according to claim 41, wherein the matrix is implanted into the damaged tissue.

43. A method according to claim 42, wherein the damaged tissue is damaged skin.

44. A method according to any of claims 41 to 43, wherein following administration of the matrix to the damaged tissue, the matrix is not manually removed or replaced.

45. A matrix as defined in any of claims 1 to 40, for use in repairing damaged tissue in a subject.

46. A matrix for use according to claim 45, comprising implanting the matrix into the damaged tissue.

47. A matrix for use according to claim 45 or claim 46, wherein the matrix is not manually removed or replaced.

48. A method comprising contacting cells with a matrix, the matrix comprising an enzyme that is able to convert a substrate to release hydrogen peroxide and a substance that includes a substrate for the enzyme.

49. A method according to claim 48, comprising seeding or impregnating the cells into the matrix.

50. A method according to claim 48 or claim 49, comprising contacting the cells with the matrix in vitro or ex vivo.

51. A method according to any of claims 48 to 50, comprising incubating the cells and the matrix in a nutrient medium, optionally following contacting the cells with the matrix.

52. A method according to any of claims 48 to 49, comprising contacting the cells with the matrix in vivo.

53. Use of a matrix as a tissue or cell scaffold, the matrix comprising an enzyme that is able to convert a substrate to release hydrogen peroxide and a substance that includes a substrate for the enzyme.

54. A matrix for use as a tissue scaffold in repairing damaged tissue in a subject, the matrix comprising a purified enzyme that is able to convert a substrate to release hydrogen peroxide and a substance that includes a substrate for the enzyme.

55. A cell culture or tissue culture comprising a matrix, the matrix comprising an enzyme that is able to convert a substrate to release hydrogen peroxide and a substance that includes a substrate for the enzyme.

56. A method of proliferating and/or differentiating cells comprising contacting cells with a matrix, the matrix comprising an enzyme that is able to convert a substrate to release hydrogen peroxide and a substance that includes a substrate for the enzyme.

57. A method according to claim 56, wherein the cells are contacted with the matrix ex vivo or in vitro.

58. A method according to claim 56 or claim 57, comprising contacting the cells and the matrix with a nutrient medium.

59. A method according to any of claims 56 to 58, comprising incubating.

60. A method according to any of claims 48 to 52, or 56 to 59, wherein the enzyme is a purified enzyme.

61. A method according to any of claims 48 to 52, or 56 to 60, wherein the substrate is a purified substrate.

62. An implant or prosthesis comprising a matrix, the matrix comprising an enzyme that is able to convert a substrate to release hydrogen peroxide and a substance that includes a substrate for the enzyme.

63. A cell culture, implant or prosthesis according to claim 55 or 62, wherein the enzyme is a purified enzyme.

64. A cell culture, implant or prosthesis according to any of claim 55, 62 or 63, wherein the substrate is a purified substrate.
